SEQUENCE LISTING (48 sequences)

SEQ ID NO: 1 - protein, 32 aa, synthetic construct
  source 1..32: note = glycomodule motif derived from the protein LCL
SSSPAPASSAPAPARSSSASPPPPPRRPPPRP

SEQ ID NO: 2 - protein, 33 aa, synthetic construct
  source 1..33: note = glycomodule motif derived from the protein GP1
PPSPAPPSPAPPSPAPPSPAPPSPAPPSPGPPS

SEQ ID NO: 3 - protein, 90 aa, synthetic construct
  source 1..90: note = glycomodule motif derived from the protein GP2
PPSPPPPTPPSPPPPSPPPPVLSPPPSPPPPSPPPPAPPPPSPPPPVPPPPSPPPPSPPPPSPPPAAASPPPSPPPPPPPSPPPPVARLP

SEQ ID NO: 4 - protein, 75 aa, synthetic construct
  source 1..75: note = glycomodule motif derived from the protein PHC21A
MPPSPPPSPPPPPPPPPPPPPPPAPPPPPPPPPPPPPPPSPPPPPPPPPPPPPPPPPFPPPPPPPPPPPPSPPPP

SEQ ID NO: 5 - protein, 74 aa, synthetic construct
  source 1..74: note = glycomodule motif derived from the protein PHC21B
PPSPPPSPPPPPPPPPPPPPPPAPPPPPPPPPPPPPPPSPPPPPPPPPPPPPPPPPFPPPPPPPPPPPPSPPPP

SEQ ID NO: 6 - protein, 20 aa, synthetic construct
  source 1..20: note = (SP)10
SPSPSPSPSPSPSPSPSPSP

SEQ ID NO: 7 - protein, 40 aa, synthetic construct
  source 1..40: note = (SP)20
SPSPSPSPSPSPSPSPSPSPSPSPSPSPSPSPSPSPSPSP

SEQ ID NO: 8 - protein, 14 aa, synthetic construct
  source 1..14: note = OLLAS tag
SGFANELGPRLMGK

SEQ ID NO: 9 - protein, 5 aa, synthetic construct
  source 1..5: note = enterokinase cleavage sequence
DDDDK

SEQ ID NO: 10 - protein, 18 aa, synthetic construct
  source 1..18: note = carbonic anhydrase 1 signal peptide
MARTGALLLVALALAGCA

SEQ ID NO: 11 - protein, 30 aa, synthetic construct
  source 1..30: note = arylsulfatase 1 signal peptide
MHARKMGALAVLAVACLAAVASVAHAADTK

SEQ ID NO: 12 - protein, 28 aa, synthetic construct
  source 1..28: note = metalloprotease gametolysin signal peptide
MSLATRRFGAAAALLVAACVLCTAPAWA

SEQ ID NO: 13 - protein, 214 aa, synthetic construct
  source 1..214: note = LC Anti-VEGF (Ranibizumab) cloned without fusion to GM
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 231 aa, synthetic construct
  source 1..231: note = HC Anti-VEGF (Ranibizumab) cloned without fusion to GM
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 15 - protein, 295 aa, synthetic construct
  source 1..295: note = HC Ranibizumab fused to (SP)20 in C-terminal including enterokinase cleavage sequence
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLDDDDKSGFANELGPRLMGKGGGSGSPSPSPSPSPSPSPSPSPSPSPSPSPSPSPSPSPSPSPSP

SEQ ID NO: 16 - protein, 278 aa, synthetic construct
  source 1..278: note = LC Ranibizumab fused to (SP)20 in C-terminal including enterokinase cleavage sequence
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDDDDKSGFANELGPRLMGKGGGSGSPSPSPSPSPSPSPSPSPSPSPSPSPSPSPSPSPSPSPSP

SEQ ID NO: 17 - protein, 268 aa, synthetic construct
  source 1..268: note = HC Ranibizumab fused to (SP)10 in N-terminal including enterokinase cleavage sequence
SPSPSPSPSPSPSPSPSPSPGGGSGGGSGGGSDDDDKEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 18 - protein, 251 aa, synthetic construct
  source 1..251: note = LC Ranibizumab fused to (SP)10 in N-terminal including enterokinase cleavage sequence
SPSPSPSPSPSPSPSPSPSPGGGSGGGSGGGSDDDDKDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 269 aa, synthetic construct
  source 1..269: note = (SP)10 N-terminal of Certolizumab HC
QAMSPSPSPSPSPSPSPSPSPSPGGGSGGGSGGGSDDDDKEVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 20 - protein, 252 aa, synthetic construct
  source 1..252: note = (SP)10 N-terminal of Certolizumab LC
MSPSPSPSPSPSPSPSPSPSPGGGSGGGSGGGSDDDDKDIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 262 aa, synthetic construct
  source 1..262: note = (SP)10 C-terminal of Certolizumab HC
QAEVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAAGGGSGGGSGGGSPSPSPSPSPSPSPSPSPSP

SEQ ID NO: 22 - protein, 245 aa, synthetic construct
  source 1..245: note = (SP)10 C-terminal of Certolizumab LC
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSGGGSGGGSPSPSPSPSPSPSPSPSPSP

SEQ ID NO: 23 - protein, 231 aa, synthetic construct
  source 1..231: note = Certolizumab HC
QAEVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 24 - protein, 214 aa, synthetic construct
  source 1..214: note = Certolizumab LC
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 6 aa, synthetic construct
  source 1..6: note = His tag
HHHHHH

SEQ ID NO: 26 - protein, 8 aa, synthetic construct
  source 1..8: note = flag tag
DYKDDDDK

SEQ ID NO: 27 - protein, 22 aa, synthetic construct
  source 1..22: note = 3x flag tag
DYKDHDGDYKDHDIDYKDDDDK

SEQ ID NO: 28 - protein, 8 aa, synthetic construct
  source 1..8: note = TEV protease
  SITE 8: note = X can be G or A
ENLYFQSX

SEQ ID NO: 29 - protein, 7 aa, synthetic construct
  source 1..7: note = His tag
HHHHHHH

SEQ ID NO: 30 - protein, 8 aa, synthetic construct
  source 1..8: note = His tag
HHHHHHHH

SEQ ID NO: 31 - protein, 9 aa, synthetic construct
  source 1..9: note = His tag
HHHHHHHHH

SEQ ID NO: 32 - protein, 10 aa, synthetic construct
  source 1..10: note = His tag
HHHHHHHHHH

SEQ ID NO: 33 - protein, 11 aa, synthetic construct
  source 1..11: note = His tag
HHHHHHHHHHH

SEQ ID NO: 34 - protein, 12 aa, synthetic construct
  source 1..12: note = His tag
HHHHHHHHHHHH

SEQ ID NO: 35 - protein, 4 aa, synthetic construct
  source 1..4: note = linker
GGGS

SEQ ID NO: 36 - protein, 8 aa, synthetic construct
  source 1..8: note = linker
GGGSGGGS

SEQ ID NO: 37 - protein, 12 aa, synthetic construct
  source 1..12: note = linker
GGGSGGGSGGGS

SEQ ID NO: 38 - protein, 16 aa, synthetic construct
  source 1..16: note = linker
GGGSGGGSGGGSGGGS

SEQ ID NO: 39 - protein, 20 aa, synthetic construct
  source 1..20: note = linker
GGGSGGGSGGGSGGGSGGGS

SEQ ID NO: 40 - protein, 5 aa, synthetic construct
  source 1..5: note = linker
GGGGS

SEQ ID NO: 41 - protein, 6 aa, synthetic construct
  source 1..6: note = linker
GGPGGP

SEQ ID NO: 42 - protein, 9 aa, synthetic construct
  source 1..9: note = linker
GGPGGPGGP

SEQ ID NO: 43 - protein, 12 aa, synthetic construct
  source 1..12: note = linker
GGPGGPGGPGGP

SEQ ID NO: 44 - protein, 15 aa, synthetic construct
  source 1..15: note = linker
GGPGGPGGPGGPGGP

SEQ ID NO: 45 - protein, 10 aa, synthetic construct
  source 1..10: note = linker
GGGGSGGGGS

SEQ ID NO: 46 - protein, 15 aa, synthetic construct
  source 1..15: note = linker
GGGGSGGGGSGGGGS

SEQ ID NO: 47 - protein, 20 aa, synthetic construct
  source 1..20: note = linker
GGGGSGGGGSGGGGSGGGGS

SEQ ID NO: 48 - protein, 25 aa, synthetic construct
  source 1..25: note = linker
GGGGSGGGGSGGGGSGGGGSGGGGS

(19) United States
(12) Patent Application Publication
(10) Pub. No.: US 2026/0274934 A1
(43) Pub. Date: Sep. 17, 2026

(54) MODIFIED ANTIBODIES AND USES THEREOF

(71) Applicant: GAT BIOSCIENCES, S.L., Barcelona (ES)

(72) Inventors: María Teresa LÓPEZ CERRO, Barcelona (ES); Olga DURANY TURK, Barcelona (ES); Cristina LÓPEZ PAZ, Barcelona (ES)

(21) Appl. No.: 19/168,527

(22) PCT Filed: Mar. 22, 2024
(86) PCT No.: PCT/EP2024/057744

§ 371 Date: Sep. 24, 2025

(30) Foreign Application Priority Data
Mar. 24, 2023 (EP) 23382279.0

(51) Int. Cl. C07K 16/22; A61K 39/00; C12N 9/88

(52) U.S. Cl. CPC C07K 16/22; C12N 9/88; A61K 2039/54; A61K 2039/542; C07K 2317/14; C07K 2317/32; C07K 2317/40; C07K 2317/522; C07K 2317/76; C07K 2317/94; C07K 2319/30; C12Y 402/01001

(57) ABSTRACT

A new class of antibody fragments (AFs) which are fused to stabilization motifs, their uses in medicine and the process for their production.

The present invention is comprised within the field of biomedicine. It specifically relates to a new class of antibodies and fragments thereof which are fused to stabilization motifs, their uses in medicine and the process for their production.

### BACKGROUND ART

Monoclonal antibodies, antibody fragments and derived fusion proteins are widely used in diagnostics and therapy to detect and treat a wide number of diseases such as cancers, inflammatory or autoimmune diseases, neurodegenerative disorders, immune diseases or rare blood diseases as well as for the prevention and treatment of solid organ transplant rejection.

The revolution of antibodies in medicine is evidenced by the growing number of antibody based products. Antibodies are generally highly specific for a particular target and thus tend to have less off-target toxicity than is seen with small molecule therapeutics.

Despite the importance of biological drugs in medicine, including antibodies and their derivatives, the biopharmaceutical industry is facing important challenges in their production (high costs and long processing times associated with mammalian cell production) and administration routes. For instance, due to their instability, and high cost of production, antibodies are delivered as intravenous infusion or subcutaneous injection dosage forms, which are associated with adverse effects such as systemic inflammatory response, infusion reactions and low patience compliance due to pain. These factors currently restrict use of these agents to patients with more severe disease.

To overcome these factors, several efforts have been taken including engineered antibodies derivatives, alternative production hosts, or complex formulations, all of them addressed to increase stability and efficiency in the specific target tissue/organ/cell.

Novel formulations that significantly stabilizes mAbs under unfavorable conditions such as low concentration or body temperature have been shown positive results with antiVEGF antibodies Bevacizumab, Ranibizumab and Aflibercept, all of them prone to diminished function once removed from their manufacturer's vial and diluted. (Giannos et al. Pharm Res. 2018; 35(4): 78).

Recombinant antibodies against tumour necrosis factor alpha (TNFα) and anti-IL23 are being developed for orally treating inflammatory bowel disease. An engineered anti-TNF modified antibody demonstrated improved permeation into the diseased tissue within the gastrointestinal (GI) tract (Nurbhai, Suhail, et al., Scientific Reports, 2019, 9, Article number: 14042, Roberts at al., Sci Rep. 2021, 11: 19422).

Antibodies to fight against infectious gastrointestinal diseases are of interest for oral delivery both in animal and humans. Fusion of antibodies or antibody fragments with recombinant moieties have shown improved tolerance to intestinal proteases and resist degradation.

Oral administration of fusion of antibody derivatives to Fc regions of mucosal IgA have proven to be effective in protecting piglets from F4 fimbriae-bearing enterotoxigenic E. coli (F4-ETEC) infection (Virdi V. et al., Nat Biotechnol. 2019 May; 37(5):527-530). Another example is the patent document US20150252100A1, which describes a fusion protein comprising an anti-enterotoxigenic Escherichia coli (anti-ETEC) VHH fused to an IgA Fc domain.

Engineered antibodies resembling sequences of colostrum bovine antibodies have shown effectiveness as oral therapeutics (Kailash C. Bhol et al., Inflamm Bowel Dis. 2013 October; 19(11): 2273-2281).

Pegylation to increase half-life (Certolizumab PEGOL) (Pasut G. et al., BioDrugs. 2014 April; 28 Suppl 1:S15-23), PASYLATION (Somayeh Mazaheri et al., Scientific Reports volume 10, Article number: 18464, 2020) or anti Albumin antibodies (Ralph Adams et al., MAbs. 2016 October; 8(7): 1336-1346) are also alternatives to increase stability of antibodies in serum.

Topical, non-invasive administration routes for biopharmaceuticals, offer potential advantages over injections owing to its simplicity of administration, high patient acceptability, and low manufacturing cost and potential local effect.

New formulations of biologics that are easy and safe to administer are highly needed to meet the growing demand of affordable biologics that are stable for non-injected delivery routes. This is especially true for those conditions that require a localized effect without systemic exposure (for instance, dermal or intestinal or respiratory diseases). Non injectable biologics would reduce systemic adverse effects, avoid drug metabolism and dilution, and will therefore reduce required doses. New antibody derivatives are continuously being generated to interact with a range of therapeutic targets. The cost-effective and efficient production of these and other antibody derivatives is crucial for their further success.

Despite research into the oral delivery of biologics has been conducted for almost a century, their current therapeutic administration remains unchanged and limited to injection. Main barrier for oral delivery is the stability of these biologics along the gastrointestinal (GI) harsh conditions. Challenges to overcome and make oral delivery of biologics a reality are improving biologic stability in the GI tract and achieving higher penetration and target delivery.

### SUMMARY OF THE INVENTION

The authors of the present invention have found that novel modified antibodies fused to glycomodule motifs are superior to non-modified antibody in terms of potency, stability, and resistance to aggregation. The inventors have shown that the addition of glycomodule motifs confers increased neutralization activity compared to non-modified antibody and makes the antibodies fused to the glycomodule motifs more stable in front of temperature and proteases.

In view of the aforementioned, a first aspect of the present invention relates to a modified antibody (hereinafter the “modified antibody of the invention”) comprising a first antibody chain comprising a VH region and a CH1 region and a second antibody chain comprising a VL region and a CL region, wherein at least one of the antibody chains is fused to at least one glycomodule motif (GM).

In a second aspect, the present invention relates to a polynucleotide (thereinafter “the first polynucleotide of the invention”) encoding the antibody chains of the modified antibody of the invention or a polynucleotide composition (hereinafter “the polynucleotide composition of the invention”) comprising a first polynucleotide encoding the first antibody chain of the modified antibody of the invention and a second polynucleotide encoding the second antibody chain of the modified antibody of the invention.

In a third aspect, the present invention relates to a vector (hereinafter “the first vector of the invention”) comprising the polynucleotide of the invention or a vector composition (hereinafter “the vector composition of the invention”) wherein each vector comprises one of the polynucleotides of the polynucleotide composition of the invention.

In another aspect, the present invention relates to a host cell (hereinafter “the host cell of the invention”) comprising the first vector of the invention or the vector composition of the invention.

In another aspect, the present invention relates to a pharmaceutical composition (hereinafter “the pharmaceutical composition of the invention”) comprising the modified antibody of the invention, the first polynucleotide of the invention or the polynucleotide composition of the invention, the first vector or the vector composition of the invention, or the host cell of the invention, and at least one pharmaceutically acceptable excipient.

In another aspect, the present invention relates to the modified antibody of the invention, the first polynucleotide or the polynucleotide composition of the invention, the first vector or the vector composition of the invention, the host cell of the invention or the pharmaceutical composition of the invention for use in medicine.

In another aspect, the present invention relates to the modified antibody of the invention, the first polynucleotide or the polynucleotide composition of the invention, the first vector or the vector composition of the invention, the host cell of the invention or the pharmaceutical composition of the invention wherein the modified antibody is a TNFα neutralizing modified antibody for use in the treatment of inflammatory diseases.

In another aspect, the present invention relates to the modified antibody of the invention, the first polynucleotide or the polynucleotide composition of the invention, the first vector or the vector composition of the invention, the host cell of the invention or the pharmaceutical composition of the invention for use in the treatment of gastrointestinal diseases.

In another aspect, the present invention relates to the modified antibody of the invention, the first polynucleotide or the polynucleotide composition of the invention, the first vector or the vector composition of the invention, the host cell of the invention or the pharmaceutical composition of the invention wherein the modified antibody is against VEGF for use in the treatment of diseases associated with undesired vascularisation.

In another aspect, the present invention relates to the modified antibody of the invention, the first polynucleotide or the polynucleotide composition of the invention, the first vector or the vector composition of the invention, the host cell of the invention or the pharmaceutical composition of the invention wherein the modified antibody is specific towards VEGF for use in the treatment of endovascular age macular degeneration, macular edema following retinal vein occlusion, diabetic macular edema, diabetic retinopathy or myopic choroidal neovascularization.

In another aspect, the present invention relates to a method, herein after “the first method of the invention” to produce the modified antibody of the invention, wherein the method comprises:
(i) growing a cell comprising the first polynucleotide or the polynucleotide composition of the invention in conditions suitable for allowing the expression of the modified antibody from the polynucleotide or from the polynucleotides of the polynucleotide composition; and
(ii) recovering the modified antibody from the culture.

In another aspect, the present invention relates to an antibody chain (hereinafter “the antibody chain of the invention”) comprising:
(i) a VH region and CH1 region; or
(ii) a VL region and a CL region;
   wherein the antibody chain is fused to one glycomodule motif (GM).

In another aspect, the present invention relates to a polynucleotide (hereinafter “the second polynucleotide of the invention”) encoding the antibody chain of the invention.

In another aspect, the present invention relates to a vector (hereinafter “the second vector of the invention”) comprising the second polynucleotide of the invention.

In another aspect, the present invention relates to a host cell comprising the second vector of the invention.

In another aspect, the present invention relates to an in vitro method, hereinafter “the second method of the invention” for the detection of an antigen of interest present in a sample which comprises:
(i) contacting the sample with the modified antibody of the invention, wherein the modified antibody is capable of specifically binding to the antigen of interest under conditions adequate for the binding of the antigen of interest to the modified antibody and;
(ii) determining the presence of the complexes containing the antigen of interest and the modified antibody.

In another aspect, the present invention relates to an in vitro method, hereinafter “the third method of the invention” for the purification of an antigen of interest present in a sample which comprises:
(i) contacting the sample with the modified antibody of the invention, wherein the modified antibody is capable of specifically binding to the antigen of interest under conditions adequate for the binding of the antigen of interest to the modified antibody and;
(ii) recovering the complexes containing the antigen of interest and the modified antibody.

### DESCRIPTION OF THE FIGURES

FIG. 1. Screening of antibody expression by direct ELISA. For the present study the plate was coated with 0.5 μg/mL human TNFα, incubated with culture media from independent clones and detected with a HRP peroxidase conjugated anti-human IgG antibody (Fab specific). Value is presented as fold increase vs wild type strain signal.

FIG. 2. Non reducing immunoblots of ranibizumab fused to GM in different positions. Equal amounts of culture media from independent clones (named c1-13) were loaded. A) detection with anti-human IgG antibody (Fab specific) of independent clones expressing AF. B) detection with anti-human IgG antibody (Fab specific) of independent clones expressing GM-AF. C) detection with anti-OLLAS antibody of independent clones expressing AFs. Full-length human monoclonal antibody (human IgG) and protein containing OLLAS tag (C+OLLAS protein) were diluted in PBX 0.1% BSA and used as controls. AF: antibody fragment, HC: heavy chain, LC: light chain.

FIG. 3. Ranibizumab and GB-AF-010 (SEQ ID NO: 15 and SEQ ID NO: 16) antigen recognition. Evaluation of antigen recognition was performed by direct ELISA: 96 well plate was coated with 0.5 μg/mL VEGF, incubated with ranibizumab (commercial, purified) or GB-AF-010 and detected with a HRP conjugated anti-human IgG (Fab specific). Results are normalized by total Fab.

FIG. 4. Non reducing immunoblots of certolizumab fused to GM in different positions. Equal amounts of culture media from independent clones (named c1-c4) expressing AF were loaded. Tested cassettes were: (SP)₁₀-certolizumab consists of (SP)₁₀ in N-terminal of certolizumab both in heavy and light chains (SEQ ID NO: 19 and SEQ ID NO: 20), certolizumab-(SP)₁₀ consists of (SP)₁₀ in C-terminal of certolizumab both in heavy and light chains (SEQ ID NO: 21 and SEQ ID NO: 22), certolizumab-HC-(SP)₁₀ consists of (SP)₁₀ in C-terminal of certolizumab heavy chain (SEQ ID NO: 21) and light chain without glycomodule (SEQ ID NO: 24), certolizumab does not have any fusion to GM (SEQ ID NO: 23 and SEQ ID NO: 24). Detection with anti-human IgG, (Fab specific). Full length human monoclonal antibody (human IgG) prepared in PBX 0.1% BSA was used as positive control.

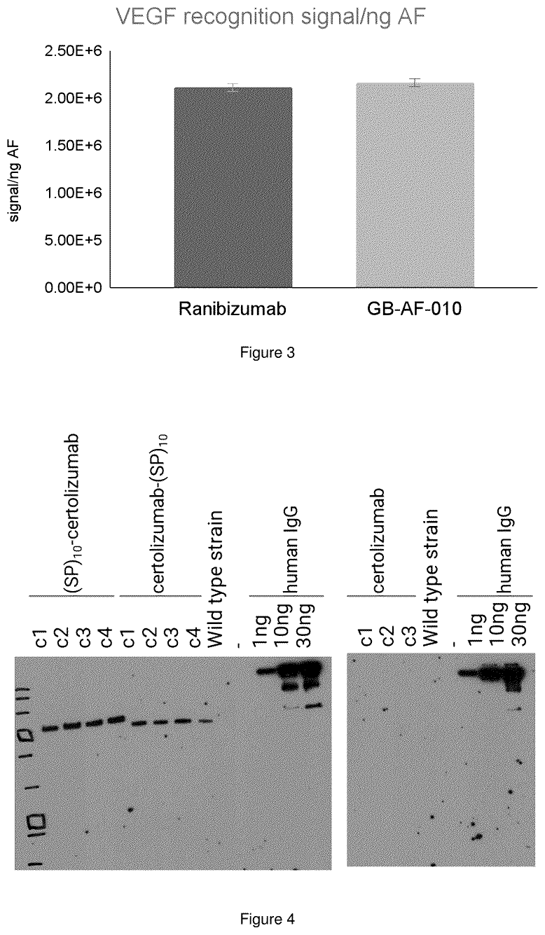

FIG. 5. Certolizumab and certolizumab-GM antigen recognition. Evaluation of antigen recognition was performed by direct ELISA: 96 well plate was coated with 0.5 μg/mL human TNFα (hTNFα), incubated with certolizumab (commercial, purified) or GB-AF-011 and detected with a HRP peroxidase conjugated anti-human IgG antibody (Fab specific). Results are normalized by total Fab.

FIG. 6. GB-AF-011 antigen recognition specificity. Evaluation of antigen recognition was performed by direct ELISA: 96 well plate was coated with 0.5 μg/mL human TNFα (hTNFα), murine TNFα or PBS as negative control (blank), incubated with GB-AF-011 and detected with a HRP peroxidase conjugated anti-human IgG antibody (Fab specific).

FIG. 7. Comparison of TNFα neutralisation capacity by several anti-TNFα agents. Evaluation of neutralisation capacity (measured as % inhibition of hTNFα binding to etanercept) was performed by competitive ELISA: 96 well plate was coated with 1 μg/mL etanercept, certolizumab (commercial, purified), infliximab (commercial, purified) or GB-AF-011 were preincubated with biotinylated TNFα, then the mixtures of biotinylated hTNFα together with anti-TNFα agent were added to the 96 well plate and detected with conjugated streptavidin-HRP.

FIG. 8. Temperature stability of GB-AF-011 compared to commercial certolizumab. Certolizumab and GB-AF-011 were incubated at 37° C. and monitored over time. Concentrations were 8 μg/mL for GB-AF-011 (lyophilized and dialyzed to PBS) and 34 μg/mL for certolizumab (purified). Results were analyzed by A) direct ELISA (average of two replicates); B) reducing immunoblot with anti-human IgG (Fab specific) for detection; C) non-reducing immunoblot with anti-human IgG (Fab specific) for detection.

FIG. 9. Stability of certolizumab-GM compared to anti-TNFα commercial references in front of colon conditions. Initial concentrations were 8 μg/mL for GB-AF-011, 32 μg/mL for certolizumab (purified), 24 μg/mL for infliximab (purified). Anti-TNFα agents were diluted 1/5 into colon content, incubated at 37° C. and collected at different times. Results were analyzed by A) reducing immunoblot with anti-human IgG (Fab specific) for detection, B) non-reducing immunoblot with anti-human IgG (Fab specific) for detection.

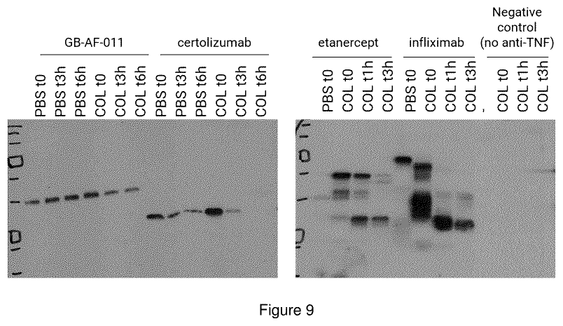

FIG. 10. Analysis of GB-AF-011 production in heterotrophic bioreactor. A) Growth monitoring of microalgae producing strain in 1 L-bioreactor. Growth was followed by optical density (OD) at 750 nm. B) non-reducing immunoblot with anti-human IgG (Fab specific) for detection. Culture media separated from cells is loaded.

FIG. 11. Novel antibody fragment structures. AF: antibody fragment. LC: light chain. HC: heavy chain. GM: glycomodule. CL: enterokinase cleavage sequence. SS-1: metalloprotease gametolysin secretion signal. SS-2: carbonic anhydrase 1 secretion sequence.

### DETAILED DESCRIPTION OF THE INVENTION

### Modified Antibodies

The authors of the present invention have discovered that modified antibodies fused to glycomodule motifs confer increased efficacy by means of improved stability and/or activity. In particular, they have discovered that modified antibodies fused to glycomodule motifs are better than non-modified antibody in terms of potency, stability, and resistance to aggregation.

Given the properties of the modified antibodies fused to glycomodule motifs, these novel antibodies will allow applications beyond injected routes and ease the use of antibodies across a number of different areas. Due to their increased stability under physiological conditions, the AFs described here, alone or in combination with other proteins such as growth factors or cytokines, can be used as a treatment for inflammatory diseases, infectious diseases, gastrointestinal diseases and/or for diseases associated with undesired vascularisation, specifically in a non-parenteral formulation (for instance oral, topical or inhaled).

Thus, in a first aspect, the invention relates to a modified antibody (hereinafter the “modified antibody of the invention”) comprising a first antibody chain comprising a VH region and a CH1 region and a second antibody chain comprising a VL region and a CL region, wherein at least one of the antibody chains is fused to at least one glycomodule motif (GM).

As used herein, a “modified antibody” refers to an immunoglobulin of any isotype, that can compete with the intact antibody for specific binding to the target antigen, and includes, for instance, chimeric, humanized and fully human modified antibodies.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

The modified antibodies of the present invention refers to: (i) antibodies that comprise complete heavy chains (VH region and CH1, CH2 and CH3 regions) and complete light chains (VL regions and CL regions) and (ii) shortened versions of an antibody which comprises a first antibody chain comprising a VH region and a CH1 region and a second antibody chain comprising a VL region and a CL region. Modified antibodies can be derived solely from a single source, or can be “chimeric” that is, different portions of the modified antibody can be derived from two different antibodies. In some embodiments, the modified antibody is an antibody fragment in which the heavy chain does not contain the constant domains CH2 and/or the constant domain CH3.

The modified antibody of the invention comprises a first chain, which refers to the heavy chain. In a particular embodiment, the heavy chain consists of a variable domain, VH, and three constant domains CH1, CH2, and CH3. In another particular embodiment of the present invention, the modified antibody only comprises the variable domain (VH) and the first constant domain (CH1). In a particular embodiment, the CH1 region is C-terminal to the VH region. In another particular embodiment, the heavy chain of the modified antibody of the invention does not comprise the constant domains CH2 and/or CH3.

The modified antibody of the invention comprises a second chain, which refers to the light chain. In a particular embodiment, the light chain consist of a variable domain, VL, and one constant domain, CL. The modified antibody of the invention comprises the VL and the CL region. The modified antibody according to the invention may comprise a complete light chain or a fragment thereof, provided the fragment comprises the VL and CL regions. In a particular embodiment, the CL region is C-terminal to the VL region.

The variable regions of heavy and light chains (VH and VL, respectively) of the modified antibody according to the invention comprise the antigen-binding sites of the immunoglobulin (Ig) molecules.

The term “antigen-binding site”, as used herein, refers to the part of the modified antibody that determines the particular antigens to which it can bind. The variable region of the heavy chain and of the light chain of the modified antibody of the invention (VH and VL) can be specified as a hypervariable because this region can bind to a wide variety of antigens. This variable region contains a region at the top which is called the antigen-binding site. The antigen-binding site is also called paratope. Each paratope is made up of six complementarity-determining regions (CDRs)—three from each of the light and heavy chains—that extend from a fold of anti-parallel beta sheets. As used herein, the term “CDR” refers to the complementarity determining region within antibody variable sequences and corresponds to an antibody region having a structure that is complementary to its target antigen or epitope.

In the modified antibody of the invention, at least one of the antibody chains is fused to at least one glycomodule motif (GM).

“A glycomodule motif (GM)”, as used herein, refers to an amino acid sequence comprising at least one residue that can be either hydroxylated and glycosylated or a residue that can be glycosylated. As used herein, the term “glycosylation site” is meant to refer to an amino acid that acts as a target site of glycosylation. In a preferred embodiment, the glycosylation site is an amino acid sequence that acts as a target for glycosylation in a microalga. Glycosylation is the reaction catalysed by glycosyltransferases, which adds carbohydrates site-specifically to another molecule, preferably proteins. Glycosylation of proteins may come in different forms, such as N-linked, O-linked and phosphoserine glycosylation. Non-limiting examples of amino acids that can become glycosylated include: proline, serine, threonine, hydroxylysine, hydroxyproline, arginine, asparagine and any variant of a natural amino acid with glycosylation potential. Thus, within glycosylation sites, proline residues may be hydroxylated to form hydroxyprolines (Hyp). In a preferred embodiment glycosylation takes place in any serine (Ser) or hydroxyproline (Pro) of the glycomodule motif. The sites for glycosylation can be placed at either or both termini of the glycomodule motif, and/or in the interior of the glycomodule if desired. Preferably, the glycosylation of the glycomodule motifs is O-Glycosylation.

Hydroxyproline O-Glycosylation is generally of two types: 1) arabinogalactan glycomodules comprise clustered non-contiguous hydroxyproline (Hyp) residues in which the Hyp residues are O-glycosylated with arabinogalactan adducts; and 2) arabinosylation glycomodules comprise contiguous Hyp residues in which some or all of the Hyp residues are arabinosylated (O-glycosylated) with chains of arabinose about 1-5 residues long. O-Glycosylation may occur following hydroxylation of one or more of the residues in the site.

In some embodiments, the modified antibody of the invention comprises only one glycomodule motif, which can be in the first antibody chain or in the second antibody chain and these glycomodule motifs can be in the C-terminal position or in the N-terminal position of the antibody chains.

Thus, in a particular embodiment, the modified antibody of the invention comprises one glycomodule motif, which is in the C-terminal position of the first antibody chain.

In another particular embodiment, the modified antibody of the invention comprises one glycomodule motif, which is in the N-terminal position of the first antibody chain.

In another particular embodiment, the modified antibody of the invention comprises one glycomodule motif, which is in the C-terminal position of the second antibody chain.

In another particular embodiment, the modified antibody of the invention comprises one glycomodule motif, which is in the N-terminal position of the second antibody chain.

In some embodiments, the modified antibody of the invention comprises more than one glycomodule motifs, in particular, two glycomodule motifs, one of them in the first antibody chain and the other in the second antibody chain. These glycomodule motifs can be in the C-terminal position or in the N-terminal position of the antibody chains.

In a particular embodiment, the glycomodule motifs are in the C-terminal position of the first antibody chain and in the C-terminal position of the second antibody chain.

In another particular embodiment, the glycomodule motifs are in the N-terminal position of the first antibody chain and in the N-terminal position of the second antibody chain.

In another particular embodiment, the glycomodule motif comprises an amino acid sequence selected from the group consisting of SEQ ID NO: 1, SEQ ID NO: 2, SEQ ID NO: 3, SEQ ID: NO 4, SEQ ID NO: 5, and a functionally equivalent variant thereof, and (SP)ₙ.

(SP)ₙ as disclosed herein refers to a nucleic acid construct that codes for n-repeating units of Serine-Proline, as disclosed in U.S. Pat. No. 9,006,410B2.

In a particular embodiment the n-repeating units is between 5 and 30. In a preferred embodiment the n-repeating units is 10 or 20. Thus, in a particular embodiment, the glycomodule motif comprises an amino acid sequence selected from the group consisting of (SP)₁₀ (SEQ ID NO: 6) or (SP)₂₀ (SEQ ID NO: 7).

“Functionally equivalent variant of SEQ ID NO: 1, SEQ ID NO: 2, SEQ ID NO: 3, SEQ ID NO: 4 or SEQ ID NO: 5”, as used herein, relates to all those sequences which result from the modification, insertion and/or deletion of one or more amino acids from the above sequence, provided that the function of the glycomodule motif is substantially maintained.

Suitable assays for determining whether a polypeptide can be considered as a functionally equivalent variant of the glycomodules would involve expressing a fusion protein comprising the glycomodule variant and a marker protein and detecting whether the addition of the glycomodule to the marker protein results in the glycosylation of the fusion protein. The presence of glycosylation in a protein can be determined by any method known in the art, including, without limitation: staining of glycoproteins (e.g. methods based on Periodic acid Schiff stain), enzymatic or chemical removal of the glycans attached to the protein and detection of the molecular weight shift by Western blot and/or mass spectrometry. A suitable assay for determining whether a given sequence acts as a glycomodule and can considered as functionally equivalent variant of the glycomodules used in the invention has been described by Ramos-Martinez et al. (Plant Biotechnol J. 2017, 15: 1214-1224).

Preferably, variants of SEQ ID NO: 1, SEQ ID NO: 2, SEQ ID NO: 3, SEQ ID NO: 4 or SEQ ID NO: 5 are (i) polypeptides in which one or more amino acid residues are substituted by a preserved or non-preserved amino acid residue (preferably a preserved amino acid residue) and such substituted amino acid may be coded or not by the genetic code, (ii) polypeptides in which there is one or more modified amino acid residues, for example, residues modified by substituent bonding, (iii) polypeptides resulting from alternative processing of a similar mRNA, (iv) polypeptide fragments and/or (v) polypeptides resulting from fusion of the polypeptide defined in (i) to (iii) with another polypeptide, such as a secretory leader sequence or a sequence being used for purification (for example, His tag) or for detection (for example, Sv5 epitope tag). The fragments include polypeptides generated through proteolytic cut (including multisite proteolysis) of an original sequence. The variants may be post-translationally or chemically modified. Such variants are supposed to be apparent to those skilled in the art.

One skilled in the art will recognize that the values of identity of nucleotide sequences can be appropriately adjusted in order to determine the corresponding sequence identity of two nucleotide sequences encoding the polypeptides of the present invention, by taking into account codon degeneracy, conservative amino acid substitutions, and reading frame positioning.

In the context of the present invention “conservative amino acid changes” and “conservative amino acid substitution” are used synonymously in the invention. “Conservative amino acid substitutions” refers to the interchangeability of residues having similar side chains, and mean substitutions of one or more amino acids in a native amino acid sequence with another amino acid(s) having similar side chains, resulting in a silent change that does not alter function of the protein. Conserved substitutes for an amino acid within a native amino acid sequence can be selected from other members of the group to which the naturally occurring amino acid belongs. For example, a group of amino acids having aliphatic side chains includes glycine, alanine, valine, leucine, and isoleucine; a group of amino acids having aliphatic-hydroxyl side chains includes serine and threonine; a group of amino acids having amide-containing side chains includes asparagine and glutamine; a group of amino acids having aromatic side chains includes phenylalanine, tyrosine, and tryptophan; a group of amino acids having basic side chains includes lysine, arginine, and histidine; and a group of amino acids having sulfur-containing side chains includes cysteine and methionine. In some embodiments of the invention, preferred conservative amino acids substitutions are: valine-leucine, valine-isoleucine, phenylalanine-tyrosine, lysine-arginine, alanine-valine, aspartic acid-glutamic acid, and asparagine-glutamine. Thus, the invention refers to functionally equivalents variants of SEQ ID NO: 1, SEQ ID NO: 2, SEQ ID NO: 3, SEQ ID NO: 4 or SEQ ID NO: 5; and that have an amino acid sequence differing in one or more amino acids with the sequence given as the result of one or more conservative amino acid substitutions. It is well known in the art that one or more amino acids in a polypeptide sequence can be substituted with at least one other amino acid having a similar charge and polarity such that the substitution/s result in a silent change in the modified polypeptide that does not alter its function relative to the function of the non-modified sequence. The invention refers to any polypeptide sequence differing in one or more amino acids, either as a result of conserved or non-conserved substitutions, and/or either as a result of sequence insertions or deletions, relative to the sequence given by SEQ ID NO: 1, SEQ ID NO: 2, SEQ ID NO: 3, SEQ ID NO: 4 or SEQ ID NO: 5, as long as said further provided polypeptide sequence has the same or similar or equivalent glycomodule motif as SEQ ID NO: 1, SEQ ID NO: 2, SEQ ID NO: 3, SEQ ID NO: 4 or SEQ ID NO: 5.

The terms “identity”, “identical” or “percent identity” in the context of two or more amino acid, or nucleotide sequences, refer to two or more sequences or subsequences that are the same or have a specified percentage of amino acid or nucleotide residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

The percentage of sequence identity may be determined by comparing two optimally aligned sequences over a comparison window. The aligned sequences may be polynucleotide sequences or polypeptide sequences. For optimal alignment of the two sequences, the portion of the polynucleotide or amino acid sequence in the comparison window may comprise insertions or deletions (i.e., gaps) as compared to the reference sequence (that does not comprise insertions or deletions). The percentage of sequence identity is calculated by determining the number of positions at which the identical nucleotide residues, or the identical amino acid residues, occurs in both compared sequences to yield the number of matched positions, then dividing the number of matched positions by the total number of positions in the window of comparison and multiplying the result by 100 to yield the percentage of sequence identity. Sequence identity between two polypeptide sequences or two polynucleotide sequences can be determined, for example, by using the Gap program in the WISCONSIN PACKAGE version 10.0-UNIX from Genetics Computer Group, Inc. based on the method of Needleman and Wunsch (J. Mol. Biol. 48:443-453, 1970) using the set of default parameters for pairwise comparison (for amino acid sequence comparison: Gap Creation Penalty=8, Gap Extension Penalty=2; for nucleotide sequence comparison: Gap Creation Penalty=50; Gap Extension Penalty=3), or using the TBLASTN program in the BLAST 2.2.1 software suite (Altschul et al., Nucleic Acids Res. 25:3389-3402), using BLOSUM62 matrix (Henikoff and Henikoff, Proc. Natl. Acad. Sci. U.S.A. 89:10915-10919, 1992) and the set of default parameters for pair-wise comparison (gap creation cost=11, gap extension cost=1).

Functionally equivalent variants of SEQ ID NO: 1, SEQ ID NO: 2, SEQ ID NO: 3, SEQ ID NO: 4 and SEQ ID NO:5 also include sequences with a sequence identity of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the sequences SEQ ID NO: 1, SEQ ID NO: 2, SEQ ID NO: 3, SEQ ID NO: 4 and SEQ ID NO: 5, respectively.

In a preferred embodiment, the functionally equivalent variant of SEQ ID NO: 1, 2, 3, 4, or 5 has a sequence identity of at least 50% with the corresponding sequence SEQ ID NO, 1, 2, 3, 4 or 5 and the sequence identity is determined throughout the whole length of the sequence SEQ ID NO: 1, 2, 3, 4 or 5.

In a particular embodiment, the first antibody chain (heavy chain) and/or the second antibody chain (light chain) of the modified antibody of the invention comprises at least one glycomodule motif, at least two glycomodule motifs, at least three glycomodule motifs, at least four glycomodule motifs, at least five glycomodule motifs, at least six glycomodule motifs, at least seven glycomodule motifs, at least eight glycomodule motifs, at least nine glycomodule motifs, at least ten glycomodule motifs or more.

In some embodiments, if the first antibody chain and/or the second antibody chain comprises more than one glycomodule motifs, all of these glycomodule motifs can be in the C-terminal position or in the N-terminal position of the antibody chains. In other embodiments, some of these glycomodule motifs can be in C-terminal position and others can be in N-terminal position of the antibody chains.

In a particular embodiment, the glycomodule motif is connected to the first antibody chain by a linker sequence.

In another particular embodiment, the glycomodule motif is connected to the second antibody chain by a linker sequence.

As it is used herein, the term “linker” means a suitable peptide that allows for two or more functional domains joined together in a fusion protein. Linkers can be flexible or rigid linkers. In a preferred embodiment the linker is a flexible linker. “Flexible linker” as it is used herein means that the joined domains require a certain degree of movement or interaction. They are generally composed of small, non-polar (e.g. Gly) or polar (e.g. Ser or Thr) amino acids. The small size of these amino acids provides flexibility, and allows for mobility of the connecting functional domains. The incorporation of Ser or Thr can maintain the stability of the linker in aqueous solutions by forming hydrogen bonds with the water molecules, and therefore reduces the unfavourable interaction between the linker and the protein moieties.

In certain embodiments, the linker is a peptide containing 1-25 amino acid residues, 1-20 amino acid residues, 2-15 amino acid residues, 3-10 amino acid residues, 3-7 amino acid residues, 4-25 amino acid residues, 4-20 amino acid residues, 4-15 amino acid residues, 4-10 amino acid residues, 5-25 amino acid residues, 5-20 amino acid residues, 5-15 amino acid residues, or 5-10 amino acid residues.

Exemplary linkers include glycine and serine-rich linkers, e.g., (GGP)n or (GGGS)n, where n is 1-5. The most commonly used flexible linkers have sequences consisting primarily of stretches of Gly and Ser residues (“GS” linker). By adjusting the copy number “n”, the length of this GS linker can be optimized to achieve appropriate separation of the functional domains, or to maintain necessary inter-domain interactions. In a preferred embodiment, the linker sequence that connect the glycomodule motif to the first antibody chain comprises (GGGS)_{n⋅} or (GGGGS)ₙ.

In another particular embodiment, the modified antibody of the invention further comprises a detection tag. As it is used herein, the term “tag” means a polypeptide useful for making the detection, isolation and/or purification of a protein easier. Generally, said labeling sequence is located in a part of the protein of interest that does not adversely affect the functionality thereof. In a more particular embodiment, the detection tag is selected from the group consisting of OLLAS tag (SEQ ID NO: 8), Flag tag (SEQ ID NO: 26) and His tag (SEQ ID NO: 25, SEQ ID NO: 29-34). In a still more particular embodiment, the detection tag is OLLAS tag (SEQ ID NO: 8).

In another particular embodiment, the modified antibody of the invention comprises a processing site between the detection tag and the rest of the chain. In a more particular embodiment, the processing site is a protease recognition site. As it is used herein, the term “protease recognition site” refers to an amino acid sequence which is susceptible to being cleaved by an enzyme that performs proteolysis, protein catabolism by hydrolysis of peptide bonds, once the protein has been translated. Suitable processing sites for use in the modified antibodies according to the present invention include an amino acid sequences that is cleavable by proteases such as enterokinase, Arg-C endoprotease, Glu-C endoprotease, Lys-C endoprotease, Factor Xa, SUMO proteases (Tauseef et al., 2005 Protein Expr. Purif. 43:1-9) and the like. In a more particular embodiment, the processing site is an enterokinase cleavage sequence (SEQ ID NO: 9) or a TEV protease (SEQ ID NO: 28), preferably is an enterokinase cleavage sequence.

The modified antibody of the invention can be derived from different antibodies. In a particular embodiment, the modified antibody derives from a neutralizing anti-tumor necrosis factor antibody (TNFα).

Tumor necrosis factor (TNFα) is a pleiotropic cytokine with beneficial functions in immune regulation and host defense, but deleterious pro-inflammatory and cytotoxic functions during inflammation. TNFα represents a critical mediator of the autoimmune process, playing a key role in several inflammatory diseases, including rheumatoid arthritis (RA), ulcerative colitis, and Crohn's disease. Inhibition of the TNFα has been achieved by the anti-TNFα biologic etanercept, antibodies like infliximab and adalimumab, or with the modified antibody certolizumab, used to treat autoimmune diseases.

In a more particular embodiment, the modified antibody of the invention derives from a neutralizing anti-tumor necrosis factor α (TNFα) selected from the group consisting of: adalimumab, infliximab, certolizumab or golimumab. In a still more particular embodiment, the modified antibody of the invention derives from certolizumab.

Adalimumab is a monoclonal antibody which is used to treat rheumatoid arthritis, psoriatic arthritis, ankylosing spondylitis, Crohn's disease, ulcerative colitis, plaque psoriasis, hidradenitis suppurativa, uveitis, and juvenile idiopathic arthritis.

Infliximab is a monoclonal antibody which is used to treat Crohn's disease, ulcerative colitis, rheumatoid arthritis, ankylosing spondylitis, psoriasis, psoriatic arthritis, and Behçet's disease.

Certolizumab is a Fab′ fragment of a recombinant humanized antibody which is used to treat Crohn's disease, rheumatoid arthritis, psoriatic arthritis and ankylosing spondylitis.

Golimumab is a monoclonal antibody which is used to treat rheumatoid arthritis, psoriatic arthritis and ankylosing spondylitis.

In a particular embodiment, the modified antibody of the invention derives from certolizumab and comprises a heavy chain as defined in SEQ ID NO: 19 or SEQ ID NO: 21 and/or a light chain as defined in SEQ ID NO: 20 or SEQ ID NO: 22.

SEQ ID NO: 19 as disclosed herein relates to a modified antibody derived from certolizumab comprising one glycomodule motif, which comprises the amino acid sequence (SP)₁₀ (SEQ ID NO: 6), and which is in the N-terminal position of the first antibody chain (heavy chain).

SEQ ID NO: 20 as disclosed herein relates to a modified antibody derived from certolizumab comprising one glycomodule motif, which comprises the amino acid sequence (SP)₁₀ (SEQ ID NO: 6), and which is in the N-terminal position of the second antibody chain (light chain).

SEQ ID NO: 21 as disclosed herein relates to a modified antibody derived from certolizumab comprising one glycomodule motif, which comprises the amino acid sequence (SP)₁₀ (SEQ ID NO: 6), and which is in the C-terminal position of the first antibody chain (heavy chain).

SEQ ID NO: 22 as disclosed herein relates to a modified antibody derived from certolizumab comprising one glycomodule motif, which comprises the amino acid sequence (SP)₁₀ (SEQ ID NO: 6), and which is in the C-terminal position of the second antibody chain (light chain).

In another particular embodiment, the modified antibody of the invention derives from a neutralizing anti-vascular endothelial growth factor (VEGF). In a more particular embodiment, the modified antibody of the invention derives from bevacizumab or ranibizumab, preferably from ranibizumab.

VEGF Vascular endothelial cell growth factor (VEGF) is a potent mitogen for vascular endothelial cells that has been reported as a pivotal regulator of both normal and abnormal angiogenesis.

Bevacizumab is a modified antibody used for colon cancer, lung cancer, glioblastoma, and renal-cell carcinoma. Ranibizumab is a modified antibody against VEGF indicated for the treatment of endovascular (wet) age macular degeneration, macular edema following retinal vein occlusion, diabetic macular edema, diabetic retinopathy and miopic choroidal neovascularization.

In another particular embodiment, the modified antibody of the invention derives from ranibizumab and comprises a heavy chain as defined in SEQ ID NO: 15 or SEQ ID NO: 17 and/or a light chain as defined in SEQ ID NO: 16 or SEQ ID NO: 18.

SEQ ID NO: 15 as disclosed herein relates to a modified antibody derived from ranibizumab comprising one glycomodule motif, which comprises the amino acid sequence (SP)₂₀ (SEQ ID NO: 7), and which is in the C-terminal position of the first antibody chain (heavy chain). The sequence SEQ ID NO: 15 comprises the enterokinase cleavage sequence.

SEQ ID NO: 16 as disclosed herein relates to a modified antibody derived from ranibizumab comprising one glycomodule motif, which comprises the amino acid sequence (SP)₂₀ (SEQ ID NO: 7), and which is in the C-terminal position of the second antibody chain (light chain). The sequence SEQ ID NO: 16 comprises the enterokinase cleavage sequence.

SEQ ID NO: 17 as disclosed herein relates to a modified antibody derived from ranibizumab comprising one glycomodule motif, which comprises the amino acid sequence (SP)₁₀ (SEQ ID NO: 6), and which is in the N-terminal position of the first antibody chain (heavy chain). The sequence SEQ ID NO: 17 comprises the enterokinase cleavage sequence.

SEQ ID NO: 18 as disclosed herein relates to a modified antibody derived from ranibizumab comprising one glycomodule motif, which comprises the amino acid sequence (SP)₁₀ (SEQ ID NO: 6), and which is in the N-terminal position of the second antibody chain (light chain). The sequence SEQ ID NO: 18 comprises the enterokinase cleavage sequence.

In another particular embodiment, the modified antibody of the invention derives from a neutralizing anti-integrin. In a more particular embodiment, the modified antibody of the invention derives from natalizumab or vedolizumab.

Natalizumab is used for the treatment of multiple sclerosis and Crohn's disease and vedolizumab is used for the treatment of ulcerative colitis or Crohn's disease.

In another particular embodiment, the modified antibody of the invention derives from a neutralizing anti-IL23/IL-12. In a more particular embodiment, the modified antibody of the invention derives from neutralizing anti-IL23/IL-12 selected from the group consisting of ustekinumab, guselkumab, tildrakizumab or risankizumab.

Ustekinumab is used for the treatment of Crohn's disease, ulcerative colitis, plaque psoriasis and psoriatic arthritis, guselkumab, tildrakizumab and risankizumab are used for the treatment of psoriasis and have potential application for Crohn's disease and ulcerative colitis.

### Polynucleotides, Vectors and Host Cells

In a second aspect, the present invention relates to a polynucleotide, hereinafter “the first polynucleotide of the invention” encoding the antibody chains of the modified antibody of the invention or a polynucleotide composition, herein after the polynucleotide composition of the invention, comprising a first polynucleotide encoding the first antibody chain of the modified antibody of the invention and a second polynucleotide encoding the second antibody chain of the modified antibody of the invention.

The terms “nucleic acid”, “polynucleotide” and “nucleotide sequence”, as used interchangeably herein, relate to any polymeric form of nucleotides of any length and composed of ribonucleotides or deoxyribonucleotides. The terms include both single-stranded and double-stranded polynucleotides, as well as modified polynucleotides (e.g., methylated, protected). Typically, the nucleic acid is a “coding sequence” which, as used herein, refers to a DNA sequence that is transcribed and translated into a polypeptide in a host cell when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence.

In some embodiments, the first polynucleotide of the invention or each of the polynucleotides forming part of the composition of the invention further comprise a nucleotide sequence encoding a secretory signal peptide wherein the secretory signal peptide is fused in frame to the N-termini of the first and second antibody chains.

As it is used herein, the term “signal peptide” or “secretory signal peptide” refers to a peptide of a relatively short length, generally between 5 and 40 amino acid residues, directing proteins synthesized in the cell towards the secretory pathway. The signal peptide usually contains a series of hydrophobic amino acids adopting a secondary alpha helix structure. Additionally, many peptides include a series of positively-charged amino acids that can contribute to the protein adopting the suitable topology for its translocation. The signal peptide tends to have at its carboxyl end a motif for recognition by a peptidase, which is capable of hydrolyzing the signal peptide giving rise to a free signal peptide and a mature protein.

Any secretory signal peptide may be used in the present invention, such as a way of illustrative non limitative example signal peptide from Chlamydomonas reinhardtii carbonic anhydrase (CAH1), having a nucleotide sequence shown in SEQ ID NO: 10, signal peptide from Chlamydomonas reinhardtii periplasmic arylsulfatase 1 (ARS1), having a nucleotide sequence shown in SEQ ID NO: 11 or the signal peptide from Chlamydomonas reinhardtii Gametolysin, having a nucleotide sequence shown in SEQ ID NO: 12.

It will be understood that, in order for the polynucleotide of the invention to be expressed in the host of cell of interest, the polynucleotide can be provided in a operably linked manner with a regulatory region. The person skilled in the art will understand that suitable regulatory regions can be used based on the host cell in which the polynucleotide can be expressed. The nature of the regulatory region is not particularly limitative in the present invention.

In another aspect, the invention relates to a vector, hereinafter “the first vector of the invention” comprising the first polynucleotide of the invention or a vector composition, hereinafter “the vector composition of the invention”, wherein each vector comprises one of the polynucleotides of the polynucleotide composition of the invention.

As it is used herein, the term “vector” or “expression vector” refers to a replicative DNA construct used for expressing the first polynucleotide or the polynucleotide composition of the invention in a cell, preferably a eukaryotic cell. The choice of expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Esherichia coli, including pCR 1, pBR322, pMB9 and their derivatives, wider host range plasmids, such as M13 and filamentous single-stranded DNA phages.

In a particular embodiment, the vector is suitable for expression in microalga. Preferred vectors for this invention are vectors developed for algae such as the vectors commonly known by the skilled person such as pChlamy_4 vector (Invitrogen), or vectors available through Chlamydomonas center.

In another aspect, the present invention relates to a host cell, hereinafter “the host cell of the invention”, comprising the first vector or the vector composition of the invention.

The term “host cell” is used such that it refers not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic (e.g., E. coli) or eukaryotic cell (e.g., yeast or plant cells).

In a particular embodiment, the host cell is a microalga. Microalga as used herein relates to a large and diverse group of simple, typically autotrophic organisms, ranging from unicellular to multicellular forms, microscopic algae, typically found in freshwater and marine systems. Examples of suitable microalgae, include microalgae from the phylums Cyanophyta, Chlorophyta, Rhodophyta, Heterokontophyta, and Haptophyta. The algae from the phylum Cyanophyta can be Spirulina (Arthrospira), Aphanizomenon flos-aquae, Anabaena cylindrica or Lyngbya majuscule. The algae from the phylum Chlorophyta can be Chlorella, Scenedesmus, Dunaliella, Tetraselmis, Haematococcus, Ulva, Codium, Botryococcus or Caulerpa spp. the algae from the phylum Rhodophyta can be Porphyridium cruentum, Gracilaria sp., Grateloupia sp, Palmaria sp. Corallina sp., Chondrus crispus, Porphyra sp. or Rhodosorus sp. The algae from the phylum Heterokontophyta can be Nannochlorropsis oculata, Odontella aurita, Phaeodactylum tricornutum. Fucus sp. Sargassum sp. Padina sp., Undaria pinnatifida, or Laminaria sp. The algae from the phylum Haptophyta can be Isochrysis sp. Tisochrysis sp. or Pavlova sp. The algae can be Chrypthecodinium cohnii, Schizochytrium, Ulkenia or Euglena gracilis. The algae can be a green microalga such as Chlorella, Scenedesmus, Dunialiella, Haematococcus and Bracteacoccus; haptophyte microalgae such as Isochrysis; and heterokontophyta microalgae such as Phaeodactylum, Ochromonas and Odontella.

In a more particular embodiment, the microalga is a green alga. Suitable examples of green alga are Chlorella or Haematyococcus, Botryococcus or Chlamydomonas. In a still more particular embodiment, the microalga is from genus Chlamydomonas.

Chlamydomonas, as used herein relates to a genus of green algae consisting of about 325 species all unicellular flagellates, found in stagnant water and on damp soil, in freshwater, seawater, and even in snow as “snow algae”. In a preferred embodiment, the microalga is from the species Chlamydomonas reinhardtii.

Chlamydomonas reinhardtii, as used herein, is a single-cell green alga about 10 micrometres in diameter that swims with two flagella. It has a cell wall made of hydroxyproline-rich glycoproteins, a large cup-shaped chloroplast, a large pyrenoid, and an “eyespot” that senses light.

In another particular embodiment, the host cell is a plant cell. The term “plant cell” as used herein refers to a plant expression system that is able to produce the glycosylation that was described in the “glycomodule motif” definition.

### Pharmaceutical Compositions

In another aspect, the invention relates to a pharmaceutical composition, hereinafter “the pharmaceutical composition of the invention”, comprising the modified antibody of the invention, the first polynucleotide or the polynucleotide composition of the invention, the first vector or the vector composition of the invention or the host cell of the invention, and at least one pharmaceutically acceptable excipient.

The term “pharmaceutical composition” is such a form that allows the biological activity of the active ingredient contained therein to be effective and has unacceptable toxicity for the subject to which the composition is administered. The term ‘pharmaceutical composition’ also covers veterinary compositions. The term “veterinary compositions” as used herein, refers to any substance or combination of substances presented as having properties for treating or preventing disease in animals or which may be administered to animals with a view to restoring, correcting or modifying physiological functions by exerting a pharmacological, immunological or metabolic action or to making a veterinary diagnosis. Pre-mixtures for medicated feeding stuffs prepared for incorporation into a feeding stuff shall also be considered to be “veterinary compositions”.

The term “excipient” refers to a substance that aids the absorption of any of the components or compounds of the pharmaceutical composition of the invention, or stabilises the components or compounds and/or aids the preparation of the pharmaceutical composition in the sense of giving it consistency or flavours to make it more palatable. Thus, excipients may have the function, by way of example, but not limited to, binding the components (e.g. starches, sugars or cellulose), sweetening, colouring, protecting the active substance (e.g. to insulate it from air and/or moisture), filling a pill, capsule or any other presentation or a disintegrating function to facilitate the dissolution of the components, not excluding other excipients not listed in this paragraph. The term ‘excipient’ is therefore defined as a material which, included in the dosage forms, is added to the active substances or their associations to enable their preparation and stability, to modify their organoleptic properties or to determine the physical and chemical properties of the pharmaceutical composition and their bioavailability.

The expression “pharmaceutically acceptable excipient”, as used herein, includes any and all solvents, dispersion media, coatings, antibacterial and antifungal agents, isotonic and absorption delaying agents that are physiologically compatible with the modified antibody of the invention, the first polynucleotide or the polynucleotide composition of the invention, the first vector or the vector composition of the invention, or the first host cell of the invention.

The “dosage form” is the configuration to which the active ingredients and excipients are adapted to provide a pharmaceutical composition or medicinal product. It is defined by the combination of the form in which the pharmaceutical composition is presented by the manufacturer and the form in which it is administered.

The pharmaceutical composition of the invention comprises the modified antibody of the invention in a therapeutically effective amount. The “therapeutically effective amount” is any amount of the component or compound of the composition which, when administered to a subject, is sufficient to produce the desired effect. Said component or compound of the composition refers to the modified antibody of the invention. The therapeutically effective amount may vary depending on, for example, the age, body weight, general state of health, sex and diet of the subject, as well as the mode and timing of administration, the rate of excretion or any possible co-treatment with other drugs.

In a particular embodiment, the modified antibody of the invention or the pharmaceutical composition of the invention is to be administered orally, topically, respiratory or by eye-drops.

The term “topically” as used herein refers to a way of administration that has local effect in a body surface, and therefore, the topical route of administration can also include enteral administration of medications that are poorly absorbable by the gastrointestinal tract, or inhaled formulations for respiratory tract delivery.

The term “enteral administration” as used herein, refers to drug administration via the human gastrointestinal tract. Enteral administration involves the esophagus, stomach, and small and large intestines (i.e., the gastrointestinal tract). Methods of administration include oral, sublingual (dissolving the drug under the tongue), and rectal administration.

The modified antibody of the invention, or the pharmaceutical composition of the invention, may be in a form suitable for oral use, for example, as tablets, troches, lozenges, aqueous or oily suspensions, dispersible powders or granules, emulsions, solutions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations.

Formulations for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Furthermore, various systems are known which can be used for sustained release administration of the pharmaceutical composition of the invention, including, without limitation, encapsulation in liposomes, microbubbles, microparticles or microcapsules and the like. Suitable sustained release forms, as well as materials and methods for their preparation, are well known in the prior art. Thus, the orally administrable form of the pharmaceutical composition of the invention is in a sustained release form further comprising at least one coating or matrix. The sustained release coating or matrix includes, without limitation, semi-synthetic or synthetic, water insoluble or modified natural polymers, waxes, fats, fatty alcohols, fatty acids, natural, semi-synthetic or synthetic plasticisers or a combination of two or more thereof. Enteric coatings can be applied by conventional processes known to the person skilled in the art.

For topical use, creams, ointments, jellies, solutions or suspensions and the like, containing the modified antibody of the present invention are employed. Similarly, transdermal patches may also be used for topical administration.

The term “respiratory administration” as used herein, refers to the delivery of drugs through the respiratory tract. It is an effective route of administration, especially for drugs with poor oral bioavailability. For respiratory use, inhalation devices, dry powder, nebulizers aerosolized liquid solutions, metered-dose inhalers (MDIs), soft mist inhalers (SMIs) and the like containing the modified antibody of the present invention are employed.

The term “eye drops” as used herein refers to liquid drops applied directly to the surface of the eye usually in small amounts such as a single drop or a few drops. The eye drops containing the modified antibody of the invention are employed. The eye drops may contain saline to match the salinity of the eye and/or a lubricant. Eye drops may be administered using an eye dropper or a glass pipette with a rubber bulb.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In addition to what is described above, the present invention also covers the possibility that the pharmaceutical composition of the invention may be administered to a subject together with other components or compounds, even if these do not form part of the pharmaceutical composition of the invention.

### Therapeutic Uses

In another aspect, the present invention relates to the modified antibody of the invention, the first polynucleotide or the polynucleotide composition of the invention, the first vector or the vector composition of the invention, the host cell of the invention or the pharmaceutical composition of the invention for use in medicine. The use in medicine referred to in the present invention may be for human or veterinary use.

In another aspect, the present invention relates to the modified antibody of the invention, the first polynucleotide or the polynucleotide composition of the invention, the first vector or the vector composition of the invention, the host cell of the invention or the pharmaceutical composition of the invention for use in medicine wherein the antibody is administered topically. The term “topically” has been described or explained above, and this definition is applicable to the therapeutic uses of the invention.

In another aspect, the present invention relates to the modified antibody of the invention, the first polynucleotide or the polynucleotide composition of the invention, the first vector or the vector composition of the invention, the host cell of the invention or the pharmaceutical composition of the invention, wherein the modified antibody is a TNFα-neutralizing modified antibody for use in the treatment of inflammatory diseases.

As used herein, the term “treating” (or “treat” or “treatment”) refers to processes involving a slowing, interrupting, arresting, controlling, stopping, reducing, or reversing the progression or severity of an existing symptom, disorder, condition, or disease, but does not necessarily involve a total elimination of all disease-related symptoms, conditions, or disorders. The treatment of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment.

The term “subject”, as used herein, refers to an individual, plant or animal, such as a human, a nonhuman primate (e.g., chimpanzees and other apes and monkey species); farm animals, such as birds, fish, cattle, sheep, pigs, goats and horses; domestic mammals, such as dogs and cats; laboratory animals including rodents, such as mice, rats and guinea pigs. The term does not denote a particular age or sex. In a preferred embodiment of the invention, the subject is a human.

In the present invention, the condition or disorder to be treated are inflammatory diseases. Inflammatory diseases include a vast array of disorders and conditions that are characterized by inflammation. Inflammatory diseases can affect the nervous system (examples include, but are not limited to encephalitis, myelitis, meningitis, neuritis, dacryoadenitis, scleritis, episcleritis, keratitis, retinitis, chorioretinitis, blepharitis, conjunctivitis, uveitis, otitis, labyrinthitis, and mastoiditis), the cardiovascular system (examples include, but are not limited to, endocarditis, myocarditis, pericarditis, arteritis, phlebitis and capillaritis), the respiratory system (examples include, but are not limited to, sinusitis, rhinitis, pharyngitis, laryngitis, tracheitis, bronchitis, bronchiolitis, pneumonitis and pleuritic), the digestive system (examples include but are not limited to, inflammatory bowel disease including ulcerative colitis and Crohn's disease, stomatitis, gingivitis, gingivostomatitis, glossitis, tonsillitis, sialadenitis/parotitis, cheilitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis and peritonitis), the musculoskeletal system (examples include but are not limited to, arthritis, dermatomyositis, myositis, synovitis, bursitis, tendinitis, panniculitis, osteochondritis, spondylitis, periostitis and chondritis), the urinary system (examples include but are not limited to nephritis, urethritis, cystitis and urethritis), the reproductive system (examples include but are not limited to oophoritis, salpingitis, endometritis, parametritis, cervicitis, vaginitis, vulvitis, mastitis, orchitis, epididymitis and prostatitis) and endocrine system (examples include but are not limited to, insulitis, hypophysitis, thyroiditis, parathyroiditis and adrenalitis).

In another aspect, the present invention relates to the modified antibody of the invention, the first polynucleotide or the polynucleotide composition of the invention, the first vector or the vector composition of the invention, the host cell of the invention or the pharmaceutical composition of the invention for use in the treatment of infectious diseases. Infectious diseases are caused by the entrance into the body of pathogenic agents or microorganisms (such as bacteria, viruses, protozoans, or fungi) which grow and multiply there. An infectious disease can differ from simple infection, which is the invasion of and replication in the body by any of various agents-including bacteria, viruses, fungi, protozoans, and worms—as well as the reaction of tissues to their presence or to the toxins that they produce. The most important barriers to invasion of the human host by infectious agents are the skin and mucous membranes. When these tissues have been broken or affected by earlier disease, invasion by infectious agents may occur. These infectious agents may produce a local infectious disease, such as boils, or may invade the bloodstream and be carried throughout the body, producing generalized bloodstream infection (septicemia) or localized infection at a distant site, such as meningitis. Infectious diseases can be caused by a virus, such as common cold, the flu (influenza), COVID-19, stomach flu (gastroenteritis), hepatitis or respiratory syncytial virus (RSV), by a bacteria, such as strep throat, salmonella, tuberculosis, whooping cough (pertussis), chlamydia, gonorrhea and other sexually transmitted infections (STIs), urinary tract infections (UTIs), E. coli or Clostridioides difficile, by fungi, such as ringworm (like athlete's foot), fungal nail infections, vaginal candidiasis (vaginal yeast infection) or thrush, and by parasites such as giardiasis, toxoplasmosis, hookworms or pinworms.

In another aspect, the present invention relates to the modified antibody of the invention, the first polynucleotide or the polynucleotide composition of the invention, the first vector or the vector composition of the invention, the host cell of the invention or the pharmaceutical composition of the invention for use in the treatment of gastrointestinal diseases. A person skilled in the art will understand that the gastrointestinal disease to be treated will require a modified antibody according to the invention which is specific for a molecule which needs to be targeted in the specific disease. For instance, if the disease to be treated is an infection by an enterotoxigenic E. coli, the modified antibody to be used will be an antibody which is specific for the E. coli fimbriae and prevents pathogen adhesion to the GI tract.

Gastrointestinal diseases refers to various disorders of the digestive system. These conditions can range from mild to severe. Some common problems include heartburn, cancer, irritable bowel syndrome, histamine intolerance and lactose intolerance. Other digestive diseases include: gallstones, cholecystitis and cholangitis, rectal problems (anal fissure, haemorrhoids, proctitis and rectal prolapse), oesophageal problems (strictures, achalasia and oesophagitis), stomach problems (gastritis, gastric ulcers usually caused by Helycobacter pylori infection and cancer), liver problems (hepatitis B, hepatitis C, cirrhosis, liver failure and alcoholic and autoimmune hepatitis), pancreatitis and pancreatic pseudocyst, intestinal problems such as polyps and cancer, infections, celiac disease, Crohn's disease, ulcerative colitis, diverticulosis, malabsorption, short bowel syndrome and intestinal ischemia, gastroesophageal reflux disease, peptic ulcer disease and hiatal hernia, among others.

In another aspect, the present invention relates to the modified antibody of the invention, the first polynucleotide or the polynucleotide composition of the invention, the first vector or the vector composition of the invention, the host cell of the invention or the pharmaceutical composition of the invention, wherein the modified antibody is a VEGF-neutralizing modified antibody for use in the treatment of diseases associated with undesired vascularisation.

Diseases associated with undesired vascularisation refers to any condition that affects the circulatory system. Some common problems related to undesired vascularisation include edema, vein occlusion, peripheral vascular disease (PVD), carotid artery disease, ischemia, abdominal aortic aneurysm, chronic venous insufficiency, deep vein thrombosis, among others.

In another aspect, the present invention relates to the modified antibody of the invention, the first polynucleotide or the polynucleotide composition of the invention, the first vector or the vector composition of the invention, the first host cell of the invention or the pharmaceutical composition of the invention, wherein modified antibody is a VEGF-neutralizing antibody for use in the treatment of endovascular age macular degeneration, macular edema following retinal vein occlusion, diabetic macular edema, diabetic retinopathy or myopic choroidal neovascularization.

### Methods for the Production of the Modified Antibodies

In another aspect, the present invention relates to a method, hereinafter “the first method of the invention”, to produce the modified antibody of the invention, wherein the method comprises:
(i) growing a cell comprising the first polynucleotide or the polynucleotide composition of the invention in conditions suitable for allowing the expression of the modified antibody from the polynucleotide or from the polynucleotides of the polynucleotide composition; and
(ii) recovering the modified antibody from the culture.

In a particular embodiment, the cell is a plant cell or a microalga. In a more particular embodiment, if the cell is a microalgae, it is a green alga, more particularly from genus Chlamydomonas, preferably from the species Chlamydomonas reinhardtii.

The first method of the invention comprises a first step of growing a cell comprising the first polynucleotide or the polynucleotide composition of the invention. The first polynucleotide or the polynucleotide composition of the invention which are comprised in the first vector or the vector composition of the invention may be introduced into the cell by means of well-known techniques such as, transfection, electroporation, via particle bombardment and transformation using the first vector of the invention that has been isolated. In a preferred embodiment the vector is introduced by transformation or electroporation. The transformed cell may be recovered on a solid nutrient media or in liquid media.

In addition, the first method of the invention comprises growing said cell in conditions suitable for allowing the expression of the modified antibody from the first polynucleotide or the polynucleotide composition of the invention. Culture conditions suitable for the growth of the microalga and for the expression of the modified antibody may be different for each type of microalga. However, those conditions are well known in the art and are readily determined.

In a particular embodiment, the microalga is cultured in a bioreactor in a suitable medium, without illumination, in mixotrophy or heterotrophic conditions, at a suitable temperature. Practically any medium suitable for growing microalgae can be used; nevertheless, illustrative, non-limitative examples of said media include TAP media. The temperature can vary usually between about 17° C. and about 37° C., particularly between 21° C. and 30° C. The culture can be performed in the absence of aeration or with aeration. Similarly, the duration of maintenance can differ with the microalga and with the amount of modified antibody desired to be prepared. Again, those conditions are well known and can readily be determined in specific situations.

The second step of the first method of the invention comprises the recovery of the modified antibody from the culture.

In a particular embodiment of the first method of the invention, the first polynucleotide or the polynucleotide composition comprises a nucleotide sequence encoding secretory signal peptides. Thus, the modified antibody is recovered from the culture supernatant.

In another particular embodiment of the first method of the invention, the modified antibodies are retained and accumulated inside the cells. Thus, the first method of the invention comprises an additional step comprising extracting the modified antibodies from the cells.

Techniques and conditions for extracting an active compound from cells are widely known in the prior art, and any of them can be employed in the context of the present invention.

“Extract” or “extraction” refers to the process by which the active compounds retained within the cell, in particular the modified antibody of the invention, are released into the medium. This extraction can be carried out by, for example, mechanical means, such as pressure or ultrasound.

Antibody Chains Modified with GMs

In another aspect, the invention relates to an antibody chain, hereinafter “the antibody chain of the invention” comprising:
(i) a VH region and a CH1 region; or
(ii) a VL region and a CL region;
   wherein the antibody chain is fused to one glycomodule motif (GM).

In a particular embodiment, if the antibody chain of the invention comprises a VH and a CH1 region, then the CH1 region is C-terminal to the VH region.

In another particular embodiment, if the antibody chain of the invention comprises a VL and a CL region, then the CL region is C-terminal to the VL region.

The term “glycomodule” has been defined or explained above, and this definition is applicable to the antibody chain of the invention. The glycomodule motif can be in the C-terminal position or in the N-terminal position of the antibody chain of the invention.

In a particular embodiment, the glycomodule motif is in the C-terminal position of the antibody chain comprising a VH region and a CH1 region (heavy chain).

In another particular embodiment, the glycomodule motif is in the C-terminal position of the antibody chain comprising a VL region and a CL region (light chain).

In another particular embodiment, the glycomodule motif is in the N-terminal position of the antibody chain comprising a VH region and a CH1 region (heavy chain).

In another particular embodiment, the glycomodule motif is in the N-terminal position of the antibody chain comprising a VL region and a CL region (light chain).

In another particular embodiment, the glycomodule motif comprises an amino acid sequence selected from the group consisting of SEQ ID NO: 1, SEQ ID NO: 2, SEQ ID NO: 3, SEQ ID NO: 4, SEQ ID NO: 5, or a functionally equivalent variant thereof, and (SP)ₙ, particularly (SP)₁₀ (SEQ ID NO: 6) or (SP)₂₀ (SEQ ID NO: 7).

In another particular embodiment, the glycomodule motif is connected to the antibody chain of the invention by a linker.

The terms “SEQ ID NO: 1”, “SEQ ID NO: 2”, “SEQ ID NO: 3”, “SEQ ID NO: 4”, “SEQ ID NO: 5”, “(SP)ₙ” and “linker” have been explained or defined above and these definitions are applicable to the antibody chain of the invention.

In another particular embodiment, the antibody chain of the invention further comprises a detection tag. In a more particular embodiment, the detection tag is selected from the group consisting of OLLAS tag (SEQ ID NO: 8), Flag tag (SEQ ID NO: 26) and His tag (SEQ ID NO: 25, SEQ ID NO: 29-34). In a still more particular embodiment, the detection tag is OLLAS tag (SEQ ID NO: 8).

The term “tag” has been defined or explained above and this definition is applicable to the antibody chain of the invention.

In another particular embodiment, the antibody chain of the invention comprises a processing site between the detection tag and the rest of the chain. In a more particular embodiment, the processing site is a protease recognition site. In a still more particular embodiment, the processing site is preferably an enterokinase cleavage sequence (SEQ ID NO: 9) or a TEV protease (SEQ ID NO: 28), preferably is an enterokinase cleavage sequence.

The term “protease recognition site” has been defined or explained above and this definition is applicable to the antibody chain of the invention.

In another aspect, the present invention relates to a polynucleotide, hereinafter “the second polynucleotide of the invention”, encoding the antibody chain of the invention.

In a particular embodiment, the second polynucleotide of the invention further comprises a nucleotide sequence encoding a secretory signal peptide wherein the secretory signal peptide is fused in frame to the N-termini of the antibody chain of the invention.

In a more particular embodiment, the signal peptide is selected from the group consisting of carbonic anhydrase 1 (CAH) signal peptide (SEQ ID NO: 10), the ARS signal peptide (SEQ ID NO: 11) or the gametolysin signal peptide (SEQ ID NO: 12).

The terms “polynucleotide” and “signal peptide” have been defined or explained above, and these definitions are applicable to the second polynucleotide of the invention.

In another aspect, the present invention relates to a vector, hereinafter “the second vector of the invention”, comprising the second polynucleotide of the invention.

The term “vector” has been defined or explained above and this definition is applicable to the second vector of the invention.

In another aspect, the present invention relates to a host cell comprising the second vector of the invention.

In a particular embodiment, the host cell is a plant cell or a microalgae cell, preferably is a microalgae cell from the species Chlamydomonas reinhardtii.

The term “host cell” has been defined or explained above.

### Methods for the Detection of Antigens in a Sample

In another aspect, the present invention relates to an in vitro method, hereinafter “the second method of the invention” for the detection of an antigen of interest present in a sample which comprises:
(i) contacting the sample with the modified antibody of the invention, wherein the modified antibody is capable of specifically binding to the antigen of interest under conditions adequate for the binding of the antigen of interest to the modified antibody and;
(ii) determining the presence of the complexes containing the antigen of interest and the modified antibody.

In the present invention the term “in vitro” means that the determination of the presence of the complexes containing the antigen of interest and the modified antibody is carried out outside the subject's body. The term “subject” has been defined or explained above, and this definition is applicable to the second method of the invention.

The term “sample” refers to a small part or quantity of a thing which is considered representative of the whole and which is taken or separated from it for the purpose of study, analysis or experimentation. In the present invention, said study, analysis or experimentation refers to the detection of the presence of complexes containing the antigen of interest and the modified antibody. The term “sample” also includes samples that have been manipulated in some way after their collection e.g., by treatment with reagents, solubilisation or enrichment of certain components. In a preferred embodiment, the sample is a biological sample.

The term “biological sample” includes, but is not limited to, biological tissues and/or fluids from an individual, obtained by any method known to a person skilled in the art for that purpose. Examples of such samples include, but are not limited to, blood samples and other liquid samples of biological origin, solid tissue samples, such as biopsy samples or tissue cultures or cells derived therefrom and their progeny, such as cells in cell culture, cell supernatants, cell lysates, serum, plasma, biological fluids and tissue samples.

The term “detect” or “detecting” refers to report or identify the presence of the complexes containing the antigen of interest and the modified antibody that are present in the sample by generating a signal.

In a first step, the second method of the invention comprises contacting the sample with the modified antibody of the invention, wherein the modified antibody is capable of specifically binding to the antigen of interest. The contact between the sample and the modified antibody of the invention has to be done under conditions adequate for the binding of the antigen of interest to the modified antibody of the invention.

Contacting the sample with the modified antibody of the invention under effective conditions and for a period of time sufficient to allow the formation of the complexes is generally a matter of simply adding the antibody composition to the sample and incubating the mixture for a period of time long enough for the modified antibodies to form the complexes with the antigen of interest.

“Under conditions adequate for the formation of a complex” means that the conditions preferably include diluting the antigens and/or modified antibodies with solutions such as BSA, bovine gamma globulin (BGG) or phosphate buffered saline (PBS)/Tween. These added agents also tend to assist in the reduction of nonspecific background.

The “suitable” or “adequate” conditions also mean that the incubation is at a temperature or for a period of time sufficient to allow effective binding. Incubation steps are typically from about 1 to 2 to 4 hours or so, at temperatures preferably on the order of 21° C. to 37° C., or may be overnight at about 4° C. or so.

The second step of the second method of the invention comprises determining the presence of the complexes containing the antigen of interest and the modified antibodies. In general, the detection of these complexes is well known in the art and may be achieved through the application of numerous approaches. These methods are generally based upon the detection of a label or marker, such as any of those radioactive, fluorescent, biological and enzymatic tags. Of course, one may find additional advantages through the use of a secondary binding ligand such as a second antibody and/or a biotin/avidin ligand binding arrangement, as is known in the art.

As the person skilled in the art will understand that there is a wide range of conventional assays that can be used in the second method of the present invention such as Western blot or immunoblot, ELISA (Enzyme-Linked Immunosorbent Assay), RIA (Radioimmunoassay), competitive EIA (Competitive Enzyme Immunoassay), DAS-ELISA (Double Antibody Sandwich-ELISA), immunocytochemical and immunohistochemical techniques, flow cytometry or multiplex detection techniques based on using protein microspheres, biochips or microarrays which include the modified antibody of the invention.

It will also be understood that modified antibodies that are not labelled need to be detected with an additional reagent, for example, a secondary antibody that is labelled, which will be labelled. This is particularly useful in order to increase the sensibility of the detection method, since it allows the signal to be amplified.

### Methods for the Purification of Antigens of Interest

In another aspect, the present invention relates to an in vitro method, hereinafter “the third method of the invention” for the purification of an antigen of interest present in a sample which comprises:
(i) contacting the sample with the modified antibody of the invention, wherein the modified antibody is capable of specifically binding to the antigen of interest under conditions adequate for the binding of the antigen of interest to the modified antibody and;
(ii) recovering the complexes containing the antigen of interest and the modified antibody.

The terms “in vitro”, “sample” and “biological sample” have been defined or explained above, and these definitions are applicable to the third method of the invention.

The phrase “specifically binding to” refers to a binding reaction that is determinative of the presence of a target antigen in the presence of a heterogeneous population of proteins and other biologics. Thus, under designated assay conditions, the modified antibodies bind preferentially to a particular antigen and do not bind in a significant amount to other components present in the sample.

In a first step, the third method of the invention comprises contacting the sample with the modified antibody of the invention, wherein the modified antibody is capable of specifically binding to the antigen of interest. The contact between the sample and the modified antibody of the invention has to be done under conditions adequate for the binding of the antigen of interest to the modified antibody of the invention.

Contacting the sample with the modified antibody of the invention under effective conditions and for a period of time sufficient to allow the formation of the complexes is generally a matter of simply adding the antibody composition to the sample and incubating the mixture for a period of time long enough for the modified antibodies to form the complexes with the antigen of interest.

“Under conditions adequate for the formation of a complex” means that the conditions preferably include diluting the antigens and/or modified antibodies with solutions such as BSA, bovine gamma globulin (BGG) or phosphate buffered saline (PBS)/Tween. These added agents also tend to assist in the reduction of nonspecific background.

The “suitable” or “adequate” conditions also mean that the incubation is at a temperature or for a period of time sufficient to allow effective binding. Incubation steps are typically from about 1 to 2 to 4 hours or so, at temperatures preferably on the order of 21° C. to 37° C., or may be overnight at about 4° C. or so.

The second step of the third method of the invention comprises recovering the complexes containing the antigen of interest and the modified antibody. Techniques and conditions for recovering these complexes are widely known in the prior art, and any of them can be employed in the context of the present invention. Some examples of techniques adequate for recovering these complexes are affinity chromatography techniques, ligand binding assays or lectin binding assays.

### EXAMPLES

The following examples illustrate the invention and must not be considered as limiting the scope thereof.

### Example 1. Design, Production and HTS Comparison of Different Antibody Fragments in Microalgae

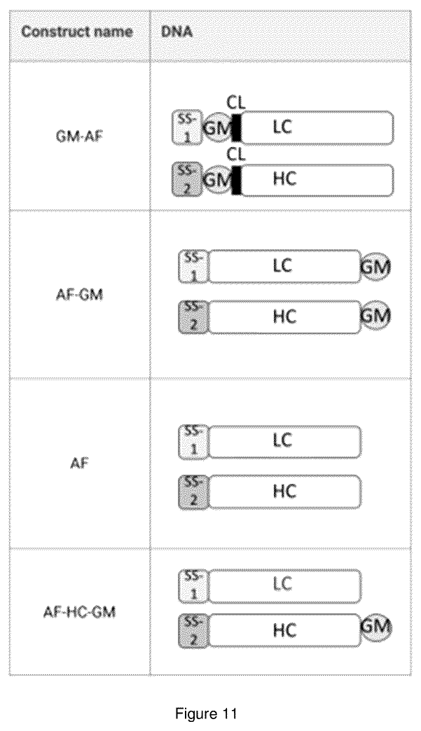

Heavy chain (VH and CH1 region) and light chain (VL and CL regions) of referenced antibody fragments (AFs) were cloned into coexpression vector (as in the vector described in the patent document WO2019215303A1). Heavy chain (HC) and light chain (LC) sequences were codon adapted to the Chlamydomonas nuclear codon AFs heavy and light chains fused with glycomodule motif (GM) of different sizes and in different positions were tested (FIG. 11). Different GMs have been considered including (SP)₁₀ (SEQ ID NO: 6), (SP)₂₀ (SEQ ID NO: 7), GP1 (SEQ ID NO: 2), PHC21A (SEQ ID NO: 4) and LCL (SEQ ID NO:1). Secretion signal sequences were cloned 5′ of the AF sequences so that the AFs are targeted to the periplasmic space or secreted to culture media and therefore are easily recovered with culture media. Signal sequences included were metalloprotease gametolysin secretion signal (SEQ ID NO: 12) or carbonic anhydrase 1 secretion sequence (SEQ ID NO: 10). The enterokinase cleavage sequence (SEQ ID NO: 9) was added to some of the expression cassettes.

Co-expression vectors (as the vector described in the patent document WO2019215303A1) carrying DNA sequences for the expression of novel AFs (as presented in FIG. 11) plus an additional hygromycin or zeocin resistance cassette, were transformed into Chlamydomonas reinhardtii by electroporation or glass bead transformation. After selection of transformants by growth on TAP plates containing zeocin, the transgenic microalgae expressing polynucleotides encoding heavy chain (VH and CH1 region) and light chain (VL and CL regions) fused to GM motifs and targeted to the periplasmic or secreted media were grown in 96 well plates. Transgenic microalgae expressing the fully assembled antibody fragment is selected by a screening method that may be dot blot, ELISA or western blot. The algae independent transformant is grown in flasks in mixotrophy or heterotrophic conditions.

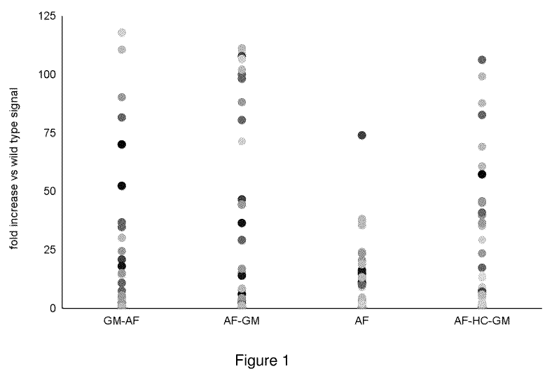

In this example screening of expression strains was performed by direct ELISA of culture media (FIG. 1). Screening results show how the use of GM fused to any of the antibody fragment chains results in increased functional AF yield.

### Example 2. Production of Fully Assembled Anti-VEGF (Ranibizumab) with and without GM

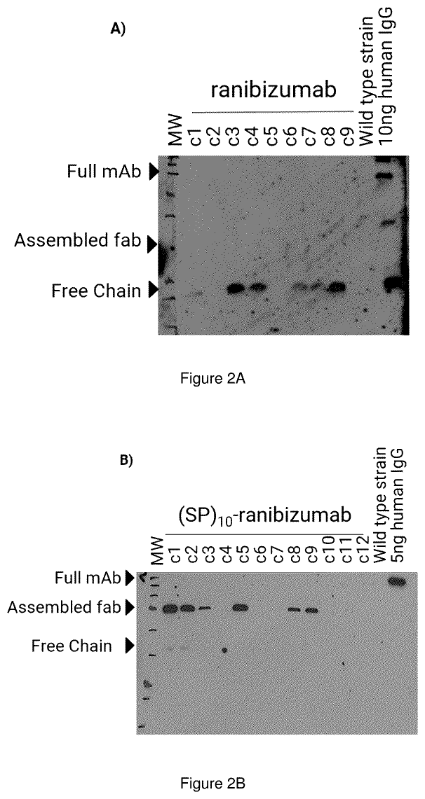

Anti-VEGF (ranibizumab) was cloned without fusion to GM (SEQ ID NO: 13 and SEQ ID NO: 14), fused to (SP)₂₀ in C-terminal of both heavy and light chains (SEQ ID NO: 15 and SEQ ID NO:16) or fused to (SP)₁₀ in N-terminal of both heavy and light chains (SEQ ID NO: 17 and SEQ ID NO: 18). Detection tags such as the OLLAS tag (SEQ ID NO: 8) and enterokinase cleavage sequence (SEQ ID NO: 9) were included in some of the cassettes. Vectors carrying DNA cassettes for the expression of these AFs plus an additional cassette for the expression of hygromycin or zeocin resistance were transformed into Chlamydomonas reinhardtii by electroporation or glass bead transformation. After selection of transformants by growth on TAP plates containing zeocin, the transgenic microalgae expressing polynucleotides encoding heavy chain (VH and CH1 region) and light chain (VL and CL regions) fused to GM motifs and targeted to the periplasmic or secreted media were grown in 96 well plates. Transgenic microalgae expressing the fully assembled antibody fragment were selected by non-reducing immunoblot of harvested culture media. FIG. 2 shows comparison of the expression of different cassettes (ranibizumab fused to GM in different positions) by non-reducing immunoblot. As it can be seen in FIG. 2, without fusion of GM there is no detectable fully assembled Fab. Furthermore, fusion of GM results in increased proportion of Fab than free chain and in increased yield of assembled Fab.

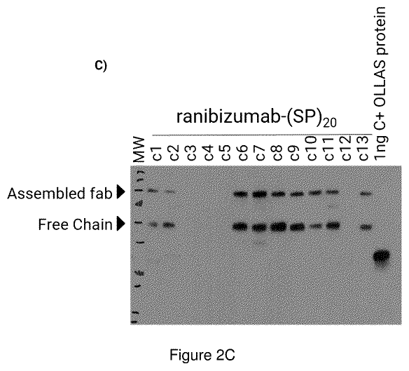

Novel ranibizumab fused to GM is named GB-AF-010 (SEQ NO ID: 15 and SEQ NO ID: 16) from here on GB-AF-010 was obtained from culture media of a transgenic microalgae expressing polynucleotides encoding heavy chain (VH and CH1 region) and light chain (VL and CL regions) fused to GM motifs and targeted to the periplasmic space or secreted to media. Activity of GB-AF-010 (non-purified, in culture media) was compared to commercial (purified) ranibizumab by direct ELISA (FIG. 3). Results show that GB-AF-010 without any purification steps, tested directly in culture media, has equal affinity for VEGF as purified commercially available ranibizumab.

### Example 3. Production of Fully Assembled Anti-TNFα (Certolizumab) with and without GM

Heavy chain (VH and CH1 region) and light chain (VL and CL regions) of certolizumab were cloned into coexpression vector (as the vector described in the patent document WO2019215303A1) fused to GM in different positions: (SP)₁₀-certolizumab consists of (SP)₁₀ in N-terminal of certolizumab both in heavy and light chains (SEQ ID NO: 19 and SEQ ID NO: 20), certolizumab-(SP)₁₀ consists of (SP)₁₀ in C-terminal of certolizumab both in heavy and light chains (SEQ ID NO: 21 and SEQ ID NO: 22), certolizumab-HC-(SP)₁₀ consists of (SP)₁₀ in C-terminal of Certolizumab heavy chain (SEQ ID NO: 21), Certolizumab does not have any fusion to GM (SEQ ID NO: 23 and SEQ ID NO: 24).

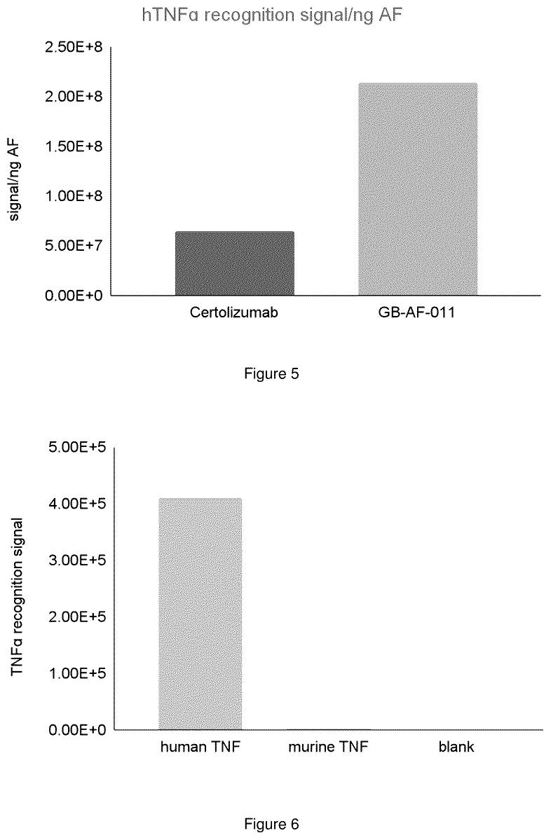

Heavy chain (HC) and light chain (LC) sequences were codon adapted. After screening by affinity by ELISA, as in Example 1—FIG. 2, several positive clones were selected to analyze expression of fully assembled AF. Immunoblot in non-reducing conditions confirmed the increased expression of Fab in constructs containing GM (FIG. 5). Low amounts of AF without GM were not enough to detect Fab by immunoblot directly from culture media.

Novel certolizumab fused to GM (SEQ ID NO: 21 and SEQ ID NO: 22) is named GB-AF-011 from here on. GB-AF-011 was obtained from culture media of a transgenic microalgae expressing polynucleotides encoding heavy chain (VH and CH1 region) and light chain (VL and CL regions) fused to GM motifs and targeted to the periplasmic space or secreted to media. The activity of GB-AF-011 was compared (non-purified) by direct ELISA with certolizumab purified from a commercially available source (FIG. 5). Results show that GB-AF-011, without any purification steps but tested directly in culture media, maintains affinity for TNFα as purified certolizumab.

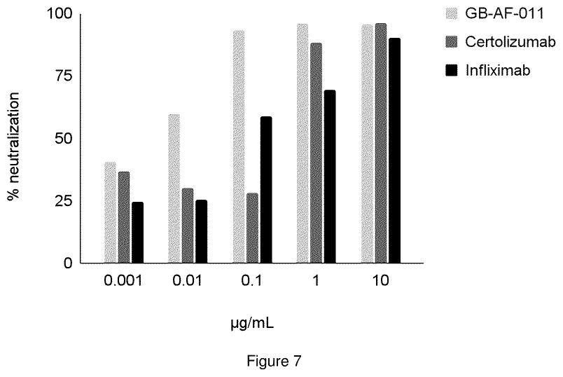

In addition, specificity of GB-AF-011 was tested by direct ELISA (FIG. 6). Results show that GB-AF-011 conserves specificity against human TNFα and does not recognize murine TNF, being comparable to non-modified certolizumab (FIG. 6).

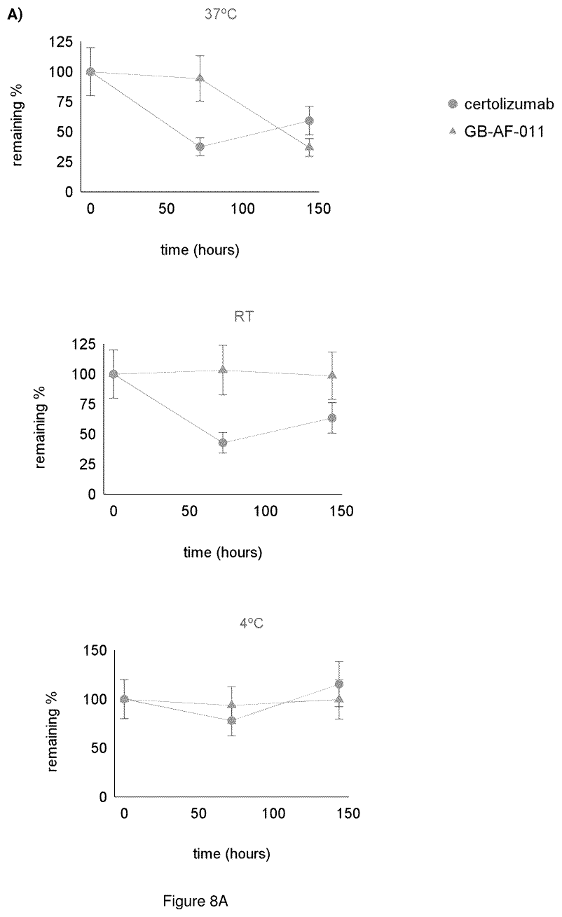

The efficacy of GB-AF-011 to neutralize TNFα was assayed by competitive ELISA, testing the ability of several anti-TNF agents to inhibit binding of labelled TNFα to its receptor (using etanercept). Surprisingly, GB-AF-011 was significantly (>10×) more effective in inhibiting TNFα binding to its receptor than commercially available purified certolizumab (FIG. 7).

### Example 4. Increased Stability of GB-AF-011 Compared to Certolizumab

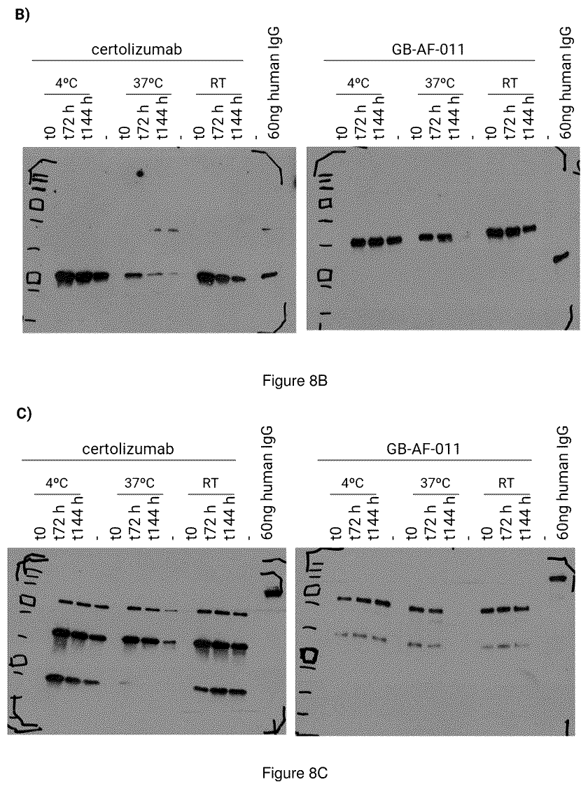

GB-AF-011 was obtained from culture media of a transgenic microalgae expressing polynucleotides encoding heavy chain (VH and CH1 region) and light chain (VL and CL regions) fused to GM motifs and targeted to the periplasmic or secreted media. GB-AF-011 was submitted to different temperatures and monitored over time and stability was compared with commercially available purified certolizumab. Results were analyzed by direct ELISA and immunoblot under both non-reducing and reducing conditions (FIG. 8). After >72 h at room temperature or 37° C. certolizumab-GM was more stable than purified certolizumab. In addition, certolizumab showed bands of higher molecular size than corresponding Fab that are associated with aggregation forms that are not observed in GB-AF-011.

### Example 5. Stability of Certolizumab-GM in Front of Colon Protease

GB-AF-011 was obtained from culture media of a transgenic microalgae expressing polynucleotides encoding heavy chain (VH and CH1 region) and light chain (VL and CL regions) fused to GM motifs and targeted to the periplasmic space or secreted to media. GB-AF-011 and commercially available purified anti-TNFα agents (certolizumab, infliximab and etanercept) were incubated under colon conditions (dilution 1/5 in colon content, 37° C.) and monitored over time. Stability of anti-TNF agents in front of colon proteases was compared by immunoblot under reducing conditions. The results show that GB-AF-011 is significantly more resistant to intestinal proteases than current therapeutically relevant antibodies.

### Example 6. Method to Produce GB-AF-011 by Fermentation

The algae expressing fully assembled Fab (GB-AF-011) was grown into a bioreactor under heterotrophic conditions until reaching high cell density. A 1 L bioreactor (PSI Photobioreactor FMT150) was used for the cultivation of C. reinhardtii capable of operating under fed-batch conditions. The bioreactor was filled with 0.9 L of a modified TAP media. The bioreactor, all connected tubing, filters, glass bottles and media were subsequently autoclaved at 121° C. for 20 min before use. A concentrated feed mimicking the basal media was prepared. The feed was filter-sterilized with a 0.22 mm vacuum cup filter and stored at room temperature. Prior to each fed-batch bioreactor culture, 200 mL of feed was transferred into a 250 mL Erlenmeyer that connected to the bioreactor. A unit was used to maintain culture temperature (21-25° C.), pH (7.30), agitation and air flow rate (0.6 L/min) throughout. Antifoam was added as required.

To prepare the Chlamydomonas strain inoculum, a shake flask containing 80 mL of TAP media was inoculated with the desired strain and grown to stationary phase on an orbital shaker at 150 rpm in mixotrophy for 3 days. 80 mL inoculum were then sterilely-added to the bioreactor pre-filled with 0.9 L of modified TAP media. The OD at 750 nm was measured to monitor culture growth and to estimate cell density. Given that the Fab is secreted directly to the culture control media to be used with or without previous purification, culture media was separated from cells and frozen at each culture time points for further analysis of Fab′ production. The fed-batch culture was grown for 8 days without illumination.

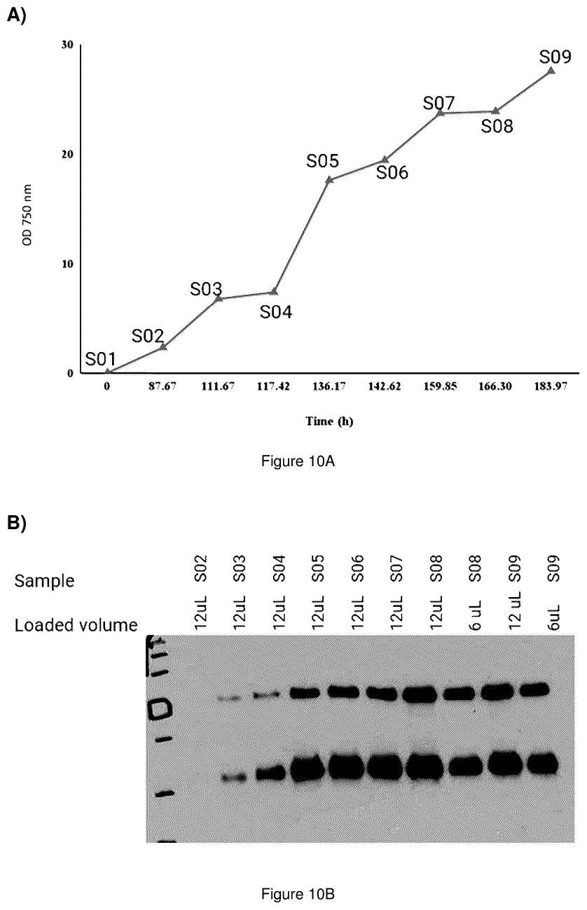

FIG. 10 shows the growth profiles of Chlamydomonas reinhardtii strains grown in a 1 L bioreactor. Production analysis of GB-AF-011 was performed by non-reducing immunoblot (FIG. 10).

1.-52. (canceled)

53. A modified antibody comprising a first antibody chain comprising a VH region and a CH1 region and a second antibody chain comprising a VL region and a CL region, wherein at least one of the antibody chains is fused to at least one glycomodule motif (GM).

54. The modified antibody according to claim 53, wherein:
(i) the modified antibody comprises one glycomodule motif and which is in the C-terminal position of the first antibody chain;
(ii) the modified antibody comprises one glycomodule motif and which is in the N-terminal position of the first antibody chain;
(iii) the modified antibody comprises one glycomodule motif and which is in the C-terminal position of the second antibody chain;
(iv) the modified antibody comprises one glycomodule motif and which is in the N-terminal position of the second antibody chain;
(v) the modified antibody comprises two glycomodule motifs and which are in the C-terminal position of the first antibody chain and in the C-terminal position of the second antibody chain; or
(vi) the modified antibody comprises two glycomodule motifs and which are in the N-terminal position of the first antibody chain and in the N-terminal position of the second antibody chain.

55. The modified antibody according to claim 53, wherein the glycomodule motif comprises an amino acid sequence selected from the group consisting of SEQ ID NO: 1, SEQ ID NO: 2, SEQ ID NO: 3, SEQ ID NO: 4, SEQ ID NO: 5, and (SP)ₙ, particularly (SP)₁₀ (SEQ ID NO: 6) or (SP)₂₀ (SEQ ID NO: 7).

56. The modified antibody according to claim 55, wherein the glycomodule motif comprises an amino acid sequence selected from the group consisting of SEQ ID NO: 2 or a functionally equivalent variant thereof or is a nucleotide sequence encoding (SP)₁₀ or (SP)₂₀.

57. The modified antibody according to claim 53, wherein the glycomodule motif is connected to the first antibody chain by a linker sequence.

58. The modified antibody according to claim 53, wherein the glycomodule motif is connected to the second antibody chain by a linker sequence.

59. The modified antibody according to claim 53, wherein the CL region is C-terminal to the VL region.

60. The modified antibody according to claim 53, wherein the CH1 region is C-terminal to the VH region.

61. The modified antibody according to claim 53, wherein the modified antibody further comprises
(i) a detection tag, preferably the OLLAS tag (SEQ ID NO: 8) and/or
(ii) a processing site between the detection tag and the rest of the chain, and wherein the processing site is preferably an enterokinase cleavage sequence (SEQ ID NO: 9).

62. The modified antibody according to claim 53, wherein:
(i) the modified antibody derives from a neutralizing anti-tumor necrosis factor antibody a (TNF α), preferably certolizumab; or
(ii) the modified antibody derives from a neutralizing anti-vascular endothelial growth factor (VEGF), preferably ranibizumab.

63. The modified antibody according to claim 53, wherein the modified antibody derives from a neutralizing anti-vascular endothelial growth factor (VEGF), preferably ranibizumab.

64. A polynucleotide encoding the antibody chains of the modified antibody according to claim 53 or a polynucleotide composition comprising a first polynucleotide encoding the first antibody chain of the modified antibody according to claim 53 and a second polynucleotide encoding the second antibody chain of the modified antibody according to claim 53.

65. The polynucleotide or the polynucleotide composition according to claim 64, wherein the polynucleotide or each of the polynucleotides of the polynucleotide composition further comprise a nucleotide sequence encoding a secretory signal peptide wherein the secretory signal peptide is fused in frame to the N-termini of the first and second antibody chains.

66. The polynucleotide or the polynucleotide composition according to claim 65, wherein the signal peptide is selected from the group consisting of carbonic anhydrase 1 (CAH) signal peptide (SEQ ID NO: 10), the arylsulfatase 1 signal peptide (SEQ ID NO: 11) or the gametolysin signal peptide (SEQ ID NO: 12).

67. A vector comprising the polynucleotide according to claim 64 or a vector composition wherein each vector comprises one of the polynucleotides of the polynucleotide composition according to claim 64.

68. A host cell comprising the vector or the vector composition according to claim 67.

69. A pharmaceutical composition comprising the modified antibody according to claim 53 and at least one pharmaceutically acceptable excipient.

70. A method for treating a disease selected from the group consisting of:
a) an inflammatory disease;
b) an infectious disease caused by a pathogen;
c) gastrointestinal diseases;
d) diseases associated with undesired vascularisation; and
e) endovascular age macular degeneration, macular edema following retinal vein occlusion, diabetic macular edema, diabetic retinopathy or myopic choroidal neovascularization,
comprising administering the modified antibody according to claim 53 to a subject in need thereof, wherein
for a) inflammatory disease, the modified antibody is against TNFα;
for b) the infectious disease caused by a pathogen, the modified antibody is against the pathogen,
for d) diseases associated with undesired vascularisation, the modified antibody is against VEGF,
for e) endovascular age macular degeneration, macular edema following retinal vein occlusion, diabetic macular edema, diabetic retinopathy or myopic choroidal neovascularization, the modified antibody is against VEGF.

71. The method for treating a subject according to claim 70, wherein the modified antibody is administered orally or by topical application.

72. A method to produce the modified antibody according to claim 53, wherein the method comprises:
(i) growing a cell comprising a polynucleotide encoding a modified antibody comprising a first antibody chain comprising a VH region and a CH1 region and a second antibody chain comprising a VL region and a CL region, wherein at least one of the antibody chains is fused to at least one glycomodule motif (GM), or a polynucleotide composition comprising a first polynucleotide encoding the first antibody chain of the modified antibody and a second polynucleotide encoding the second antibody chain of the modified antibody, in conditions suitable for allowing the expression of the modified antibody from the polynucleotide or from the polynucleotides of the polynucleotide composition; and
(ii) recovering the modified antibody from the culture.
